SEQUENCE LISTING (37 sequences)

SEQ ID NO: 1 - DNA, 1549 bp, Bacillus megaterium DSM 32963
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 2 - DNA, 1549 bp, Bacillus megaterium DSM 32963
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 3 - DNA, 1188 bp, Bacillus megaterium DSM 32963
atgaagaatggatggacaaactttgttattggtaccgtacgtattcgaattgttggaaaaggaattgaacgttttttaaataattgtgtgagacaacagattatgatttcgaatgtccataaagttgacggtcaactagcaacagcaacgatattattgaaagatgtaaagaaaattcgtatactaattcgcaacgcagattgtaaaatttattttataagaggcagaggttttccttttttaacaaaacgagtgatcaaaaatagtggatttgctttaggttttctttctttttttattattcttggtttgctttccaacatggtgtggaaagtggaaattagcggtgcagaaccacaaacagaacatcaaatgactaagcagcttgccaaaataggtgtaaaaagaggagagtttcaatttctgttagaaagtcctgaaaaaattcagcgctatttaacggataatatgaacaatattacctgggtgggagtagaagtgcgcggcacttcttatcacttccaagtggttgaaaaaaatgagcccaagcctcagcaaaaaacaccttaccaacatttgattgctaaaaagaaagccattattacgaatttgtttgtcgaaaaaggacaacctttagtcaaggttaatgattttgtcaatgaaggcgaggtcctagtttccggtattattggaaacgaaaaaaataaaaaggtagtagctgctaaagggaaagtctacggtgaaacatggtataagtctgaggtggaagtgcctttaaaaactgattttcaggtattaacgggtaatggatatacgaagcactatttagattttcaagcttttaaaatgcctctttgggcttttaacaaagaaaaatacgcctcaaaggtgacagaaaaagtggagcatccactctattttttaaaatggaaattacctctgtcgtatgagaaagttgctgtacgagaagaacaaaactcgcagcgcgtctattctaaacaagaggcggtggaaaaagctttggaaatcggtcgaaaaaaattactatcaactttaggtgaggattctaaaataaaaggtgaaaaagttttgcaccaagaacaggacaatggtaaagttagattatcaatacattaccaagttatagaaaatattgcgaacactcaaccaattattcaaggagattga

SEQ ID NO: 4 - DNA, 1932 bp, Bacillus megaterium DSM 32963
gtgatacatttgacgatggaacaaaaagaagtacaagcatatgaagctgatcagatacaagtattagaaggattagaggctgttcgtaaacgtccggggatgtatattggatcgacgagcgcaaagggtttacatcatcttgtatgggaaattgtagataatagtattgatgaagcgctggccggctattgcgatgaaattaatgttattatcgaaaaggataatagtattacagtcaaagataacggtcgtggaattccggttggtattcaagaaaaaatgggcagacctgccgttgaagttatcttaacggttcttcatgccggaggtaaatttggcggcggcggctataaagtatccggtggattacatggtgtaggtgcctcagttgttaacgcactttctacctctttggaagtgtacgtacatcgtgatggtaaagttcattatcaaaaatatgaacgaggtgtaccggctgctgacttaaaagtagttggagaaacagataaaacaggtactgttattcaattccatccagacggtgaaatttttacagaaacgcttgaatacgattttgatacgttagctaatcgtctgcgtgagttagctttcttaaatcgcggtattaaaattacgattgaagacaaacgtgaagaagataaaagacgtgagtatcactatgaaggcggaattaagtcttacgttgaacacttaaaccgtgcaaaagaagtaattcacgaagagccgatctatattgaaggtaatcgagacaacatttctgtagaaattgctattcaatataacgatagctatacaagtaatttatattcttttgcaaacaacattcacacatatgaaggtggaacgcacgaagcaggatttaaaacagcgttaacgcgtgtaattaacgactatgcacgtaaaaacagcgtatttaaagacagtgatgccaatctaacgggtgaagatgttcgtgaaggaattacagctatcatctctattaagcacccagatccgcagtttgaaggacaaacaaaaacaaagctgggaaatagtgaagcaagaacaattactgactctgtgtttgcagaacacttagaaacgtacttgctagagaaccctattgtggcgaaaaaggtaattgaaaaaggtttaatggctgcaagagcaagaatggcagctaaaaaagctcgtgagcttacaagacgtaaaagcgcgcttgaaatctcaaacttaccgggtaaattagcagattgttcatcaaaagatccttctattagcgaactctatgtagtagagggtgactctgcgggaggatcagctaagcagggaagaagccgtcatttccaagctattttgcctttacgtggtaaaattatcaacgtagagaaagcccgtttagataaaattttatctaataacgaaattcgtacaatcattaccgctctaggaacgggtattggtgacgattttgatatctcgaaagcacgctaccataaaattgtgatcatgacagatgcagacgtggacggtgcgcatattcgtacgcttcttctaacgttcttctatcgctatatgagacagattattgagcatggatatgtgtacattgcccagccgcctctttacaaagtttcacaaggtaaaaaagtggagtacgcgtacaacgatcgtcaattagaagaggtattagcttctttccctgaaggcgcaaaaccaaaccttcagcgttacaaaggtttaggagagatgaatcctgaacaattatgggaaacaacaatggatccagagttccgtacccttcttcaagtgaacttgcaagatgcaattgaagctgatgagacatttgaaattttaatgggcgacaaagtagaaccacgccgtaatttcattgaagaaaatgctcagtacgtaaaaaatcttgatatttaa

SEQ ID NO: 5 - DNA, 3573 bp, Bacillus megaterium DSM 32963
gtgaatcagttgacaggtcaactagttcagtatggacgccaccgccaacgcagaagttatgctcgtattagtgaagttttagagttaccgaacttaatcgagattcaaacggcttcataccaatggtttttagatgaaggtttacgagaaatgttccaagatatttctccgattgatgattttacaggtaacttatcactagaatttattgattacagcttaggtgagccaaagtattcagtaggagagtccaaagaacgtgatgtaacttatgcagcacctcttcgtgttaaggtgcggttaattaataaagaaacaggtgaagtaaaagaccaagatgtgttcatgggagatttcccattgatgaccgaaacaggtacctttgtgattaatggtgctgagcgtgttatcgtatcacagttggttcgttcaccaagtgtttactatagtggaaagcttgataaaaacggtaaaaaaggatatacagcaactgttatccctaaccgtggtgcatggctagaatatgaaactgatgcaaaagatgtagtatacgtgcgtattgatcgtactcgtaaactgccagtaacagtgctgttacgcgcgctaggtttcggttctgaccaagagattatcgatttaatcggtgataatgaatacatccgtaatacgcttgaaaaagataatacggaaacaacggaaaaagcgctattagaaatctatgagcgtttacgtccgggtgaaccaccgacagttgagaatgcgaagtctctattagtatctcgcttctttgatccaaaacgatatgacttagcgaacgtaggtcgctataaaattaataaaaagcttcatatcaaacatcgcttatttaatcaaaagctagctgaaacattagttgatcctgaaacaggtgaaattattgcagaaaaaggcgcaatgattgaccgccgtctgctagatcgcttgattccgatgcttgaaggtggagtaaacttcaaaacttatagtccggttggtggagtagtagaagacgatgttacattacaatctattaagatttatgcgccaaatgatccagaaggtgaaaaaatcatcactgtatcaggtaacgcatatgtaacagaagaagttaaaaatatcacacctgctgatattttagcatcaattagttacttctttaacttgcttcatcaagtaggagacacagatgatatcgaccacttaggtaaccgtcgtctgcgttctgtaggtgaattgttacaaaaccaattccgtatcggtttatctcgtatggaacgtgttgttcgtgaaagaatgtcaattcaagacacgaatacaatcacacctcaacaattaattaatattcgcccagttattgcggcgattaaagagttctttggaagttctcaattatcacagttcatggatcaaacgaatccattaggcgaattgacgcacaaacgtcgtctttcagctctaggacctggtggtttaacgcgtgagcgcgctggttttgaagtgcgtgacgttcactactcccactatggccgtatgtgtccgattgaaactccagagggtccgaatatcgggttaatcaactcactatcttcttatgcaaaagtaaacaaattcggtttcatcgaaacaccttaccgtcgtatcgatcctgaaacaggtaaagtgacagagcgaattgactacttaacagctgatgaagaagataactatgttgtagcccaagcgaacgctcgtctaggcgatgatggttcattcttagatgaaaatgtcgttgcacgtttccgtggagaaaacacggttatccgtcgcgatcgtttagattatatggatgtatcaccaaaacaagttgtatctgccgctacagcatgtatcccattcttagagaacgatgactctaaccgtgcattaatgggtgcgaacatgcaacgtcaagcagtacctttattaaatcctgaagcaccaatcgtaggtacaggtatggaatacgtatctggtaaagactctggtgcagccgtgatttgtaaatatcctggcgttgtagagcgcgtagaagcaaaacaaatttttgttcgccgctatgaagaagtagacggacaaaaagttaaaggtaacttagatcaatacaaattattaaaatttgttcgttctaaccaaggtacttgttacaaccagcgtccaattgtttcagttggcgacgaagtagtaaaaggtgagatcttagccgacggtccttcaatggaaaaaggtgagcttgctttaggacgaaacgtaatggttggtttcatgacatgggatggttacaactatgaggatgccatcatcatgagtgaacgccttgtgaaagatgatgtatatacgtctgttcatattgaagaatatgaatctgagtctcgtgatacgaagcttggacctgaagaaattacgcgtgacattccaaacgtaggtgaagatgcgcttcgcaacttagatgagcgtggaatcatccgcattggtgcagaagtaaaagacggagatcttttagttggtaaagtaacgccaaaaggtgtaacagaactaacagctgaagaacgtcttctacacgctattttcggtgaaaaagcgcgtgaagttcgtgatacttctcttcgtgtaccgcacggcggcggtggaatcattcttgatgttaaagtcttcaaccgtgaagatggcgacgaattaccaccaggtgtaaaccaattagtccgtgtatatattgttcagaagcgtaaaatttctgaaggtgacaaaatggccggtcgtcacggtaacaaaggtgtaatttcacgtattttacctgaagaagatatgccttatctaccagacggtacgccaattgacatcatgttaaacccattaggggtaccatctcgtatgaacatcggtcaggtgctagagcttcatttaggtatggctgctcgtaagcttggcattcacgttgcgtctccagtatttgatggtgcgcgtgaggaagatgtgtgggcaacaatcgaagaagctggcatgtctcgtgatgctaaaacagttctatatgatggtcgaacaggtgaaccattcgataaccgtgtatcagtaggaatcatgtacatgatcaaacttgctcacatggtagacgataaacttcacgctcgttctactggaccatactcacttgttacacaacaaccacttggtggtaaagcgcagttcggtggacagcgttttggtgagatggaggtatgggcacttgaagcatacggtgctgcttacacattacaagagatcttaacagtgaaatcagatgacgtagtaggtcgtgtgaaaacatacgaagcaattgttaaaggtgaaaacattccagaacctggcatacctgaatcgttcaaagtattaattaaagaactacaaagtttaggtatggatgtgaagatgctttctgctgacgagcaagaaattgatatcatggactcagaagaggaccatgagcaaccaacagaatcaattattgctgataacgaagaaagcctttctgaaggacaaaaagatcctgtcacaaaagagtaa

SEQ ID NO: 6 - DNA, 1632 bp, Bacillus megaterium DSM 32963
atggcaaaagacattaaatttagcgaagaagcacgtcgcgcaatgctacgtggtgtagacacattggcaaatgctgtaaaagtaacgcttggaccaaaaggtcgtaacgttgtattagaaaagaaattcggttcaccgcttattacaaatgacggtgtaacaattgcaaaagaaatcgaattagaagacgcatttgaaaatatgggtgctaaattagtagccgaagttgctagcaaaacaaacgacgtcgctggtgacggtacaactactgcaacagttttagcgcaagcaatgatcagagaaggtcttaaaaacgtaacggctggtgctaacccaatgggtatccgtaaaggtatggaaaaagcagtagctgtagcggttgaagaactaaaagcaatctctaaaccaattcaaggtaaagattcaattgctcaagtagcggctatctcagcagctgacgaagaagtaggtcaattaattgctgaagcaatggagcgcgttggtaacgacggcgttatcacacttgaagaatcaaaaggtttcacaactgaattagaagtggtagaaggtatgcagtttgaccgtggatatgcatctccttacatggtaactgattcagataaaatggaagctgtattagatgatccatacatcttaatcacagacaaaaaaatcggtaacattcaagaaatcttaccggtattagagcaagttgttcaacaaggcaagcccctattaatcatcgctgaagacgtagaaggcgaagctttagcaacattagttgtgaacaaacttcgtggtacattcacagctgtagctgttaaagctcctggtttcggtgatcgtcgtaaagcaatgctacaagacgttgcgatcttaacaggcggagaagtaatcactgaagagcttggtctcgacctaaaaacagcaagcattgatcaattaggtcgcgcttctaaaattgttgtaacaaaagaaaatacaacggttgtaaacggtgcaggaaacgcagaagacatcctagcacgcgtaaaccaaatcaaagctcagcttgaagaaacaacttcagagtttgaccgtgaaaaattacaagagcgcttagcaaaacttgctggtggcgtagctgtaattaaagtgggtgcggcaactgaaactgagttaaaagaacgtaaattacgtattgaagatgcattaaactctacgcgtgctgcggttgaagaaggtatcgtagctggtggtggtactgcattagtaaatatctataataaagtagcaagcatcgaagctgacggtgacactgctacaggtatcaacattgtattacgtgcgatcgaagagcctgtacgtcaaatcgctcacaatgctggtttagaaggatcagtaatcgttgagcgtctaaaaggcgaagcagttggaactggattcaatgctgcaactggcgagtgggtaaacatgctagacactggtatcgttgacccaacaaaagtaacgcgttcagctcttcaaaatgcttcttctgtagcggctatgttcttaacaactgaagcagttgttgctgacaagccagaagaaggcggagcacctgcaatgcctgacatgggcggcatgggtggaatgggcggcatgatgtaa

SEQ ID NO: 7 - DNA, 3150 bp, Bacillus megaterium DSM 32963
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcacgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcgtatgctaaacggaaaagatccagaaacgggtgagccgcttgatgacgagaacattcgctatcaaattattacattcttaattgcgggacacgaaacaacaagcggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtagaagagttccgcccagagcgttttgaaaacccaagcgcaattccgcagcatgcgtttaaaccatttggaaacgggcagcgcgcgtgtatcggtcagcagttcgctcttcatgaagcaacgcttgtacttggtatgatgctaaaacactttgattttgaagatcatacaaactacgagctggatgttaaagaaactttaacgttaaaacctgaaggctttgtggtaaaagcaaaatcgaaacaaattccgcttggcggtattccttcacctagcactgaacagtctgttaaaaaagtacgcaaaaaggtagaaaacgctcataatacgccgctgcttgtgctatacggttcgaacatgggaacagccgaaggaacggcgcgtgatttagcggatattgcgatgagcaaaggattcgcaccgcaggtcgcagcgcttgattcccacgcaggaaatcttccgcgcgaaggagctgtattaattgtaacggcgtcttataacggacacccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttattgatgaaacgcttgccgctaaaggagcagaaaacatcgctgaacgcggtgaagcagatgcaagcgacgactttgaaggcacatatgaagaatggcgtgaacacatgtggagtgacgtagcagcctactttaacctcgacattgaaaacagtgaagataataaatctacgctttcacttcaatttgtcgacagcgctgcggacatgccgcttgctaaaatgcacggtgcgttttcagcaaacgtcgtagcaagcaaagaacttcaacagccaggcagtgaacgaagcacgcgtcatcttgaaattgaacttccaaaagaaacttcttatcaagaaggagatcatttaggcgttattcctcgcaactatgaaggaatagtaaatcgtgtgacgacaagatttggtctagatgtatcacagcaaatccgcttggaagcagaagaagaaaaattagcgcatttgccactcggtaaaacagtatcagtagaagagcttctgcaatacgtagagcttcaagaccctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatggaattcagcgaatttatcgcccttctgccaagcatacgcccgcgctattattcaatttcatcttcacctcgtgtcgatgaaaaacaagcaagcattacggtcagcgttgtctcaggagaagcgtggagcggatacggagaatacaaaggaattgcgtcaaactaccttgccgagctgcaagaaggagatacgattacgtgctttgtttctacaccgcagtcaggatttacgctgccaaaagaccctaaaacgccgcttatcatggtcggaccgggaacaggcgttgcaccgtttagaggctttgtgcaggctcgcaagcagctaaaagaacaagaagagtcgcttggagaagcgcatttatactttggctgccgttcacctcatgaagactatctgtatcaagaagagcttgaaaacgcccaaaatgaaggcatcattacgcttcataccgctttttctcgcgtaccaaatcagccgaaaacatacgttcagcacgtaatggaacaagacggcaagaaattgattgaacttcttgaccaaggagcgcacttctatatttgcggagacggaagccaaatggcacctgacgttgaagcaacgcttatgaaaagctatgctgacgttcaccaagtgagtgaagcagacgctcgcttatggctgcagcagctagaagaaaagggccgatacgcaaaagacgtgtgggctgggtaa

SEQ ID NO: 8 - DNA, 1812 bp, Bacillus megaterium DSM 32963
gtgttgcaacttaaggtagtaaacagcccttttaatcaagaacaagcagatttgcttaatcgccttctgccgacattaacagaagcacaaaaaatgtggttgagcggttatttaacagcagctcaatctacgtctgccgaaggaacgccagacgtttctacagcagcgcctgctcaaacgaaacagacagtttcaaaagacgtaacgattctttatggatcacaaacaggaaatgctcaaggacttgctgaaaatacaggcaaaacgcttgaagcaaaaggctttaatgtaactgtatcttctatgaatgatttcaaaccaaataatttaaagaaacttgaaaatttattaattgtcgtaagtacacatggagaaggagagccgcctgataatgcgctatctttccatgaatttcttcacggccgtcgagcgccaaaacttgagaacttccgtttctctgttttatcgcttggagacagctcatacgaatttttctgtcaaacaggaaaagaatttgatgtgcgcttagcagaacttggcggtgaaagactgtatccgcgcgttgactgtgatttagattttgaagagcccgcaaataaatggcttaaaggtgttattgacggattaagcgaagcgaaaggacacagcgcttcggcagctgttccagcggaagctcctgcaggaacttcaccatattcaagaacaaacccttttaaagcagaagtgcttgagaacttaaacttaaacggccgcggatcaaataaagaaacgcgacacttagaactatctctagaaggttcaggtttgacgtatgaaccaggagacagtttaggtatttatcctgaaaatgacccggagcttgttgatcttcttcttaacgaattcaagtgggatgcaagtgaaagtgtaacggttaataaagaaggagaaacgcgtcctcttagagaagcgctaatctctaattttgaaattaccgttttaacaaagccgcttttaaagcaagcagctgagcttactggaaacgataagttaaaagcgcttgtagaaaatcgcgaggaattaaaagcatacacacaaggccgtgatgtaattgacttagttcgtgacttcggtccatggaacgtatcggcacaagagtttgtagccattctacgtaaaatgccagcgcgtctttactcgattgcaagcagcttatcagcaaaccctgatgaagttcatctaacaatcggagcggtacgctacgaagcgcatggacgcgagcgtaaaggtgtttgttcagtcctttgttcagaacgtttgcagccaggcgatacgattcctgtataccttcaaagcaataaaaactttaagcttcctcaagatcaagaaacgccaattattatggtaggacctggtacaggtgtggctccgttccgctcatttatgcaagagcgtgaagaaacaggggcaaaaggaaagtcatggatgttctttggagatcagcacttcgtaacagacttcctttaccaaacagaatggcaaaagtggttaaaagatggcgtactgacaaaaatggacgtggcgttttcacgcgatacagaagaaaaagtatacgtacaaaaccgtatgctcgaacatagtaaagaattattccaatggttagaagaaggcgcatctttttatgtgtgcggagataaaacaaatatggcacgcgacgtgcacaacacgctagttgaaattatcgaaacagaaggcaagatgagccgtgaacaggcggaaggttaccttgctgaaatgaagaaacaaaaacgttatcagcgtgatgtatactga

SEQ ID NO: 9 - DNA, 1278 bp, Bacillus megaterium DSM 32963
ttgccggttatacagggcccctcgtcgtataagcttaccggtcacctgcaaaagtttagagagaaccccctgggttttttggaaaacttgactcagtatggagaaattgcaacatttagagttgcgcacaaacgcttttatgtaacgagagaccctcaacttattaaagatgtagtcattacaaacagcaaggcgtttcaaaaaattaaactaacgcatatgtttaaaacgctgctaggcgaagaaatgttatggacagatgaagcattatatatgagccccattcaaccctcacaactaaaacaacatctaacttttaataaggaagcaatcgcaaaaataattgaaaagcatacggaaacgtgggaagaagggcagcttcgaacaatagtaaaagacataagacagatagtcatagctgtactccttcaacttgtatttggcatttcgattgaggagaaagataaaattcactatgttcaagcgcttatgagaaaaaaagaaaagttaggcaaaatttatattcgtctacctttgcaccagcctgactcagatgagcagctagaacaactcctttttgaacgcgttcaaatgcgtgttcaaaacaaaaccgcaggaaatgatttactgcagtatatattgaattcttacggagaagacagcgatgaaagagaaatatacgaacagctaaattctatattcctttcgatgtatgaaatgattacgcacgtatgcagctggtctattcatttactatctcaaaatactagggagcaccttcagctgcataaagaaattcaagcctatgctagcggcgaatcactttcaaccaaaaatctgacctatatgcgcaaaataattgctgaaagcatgaggttgtatccgcctctttggctatttgggcgtcaagcacgtgaagatatacaaatagatggctacagtattaagaaaggggagattatgttaattagcccttatatgatgcatcgccatgaagattattttttagagccaagtgaatttttacctgaccgttttgaaaaaggagggtccattgatgtcccaagctatatgtatatgccacttgggattgagcatcaagcggaaaggggcatggactatatcactgaaatagtcaccatttttctgtcagaaatgacaaagcgctttttatttcagctgactaagcccgaatctattgctccgatggcgggcgtaatgctgaatatgaaagaagaactgaaggtgaatgttcacaaagttcacgcgcagtcttga

SEQ ID NO: 10 - DNA, 1215 bp, Bacillus megaterium DSM 32963
atgaaaacagaaagagaaaacggaattgtccgtcaagtgaatacgattcaaacaaaagaagaacgctttaatcctttttcatggtatgaagagatgagaaacaccgagcctgtgcaatgggatgaaaaaaggcaggtatgggatgtttttcactatgacggggtcaaagaagtgctagaacaaaagaatattttttcttctgatcgaagacctccacaaaaccaaagacaaactgctctaggaacgagcctaattaatattgatccgcctaagcatgctgaaatgagagcgcttgttaataaagcttttacgcctaaagcaatgaaagcatgggagcctaaaattgcgcgtattacaaatgaattattacaagaagttgagcatcttgaagatattgatatagtcgagcatctgtcctatccgcttccggttatggtaattgccgatattttaggggtgccgatagaagatcagcgtcagtttaaagattggtcggatattatcgtagccggtccttctaataacgaacgtgaaacgctcgaaaaactccagcaagagaaaatgaaagcaaatgatgagctagaaacttacttttatcgaatcattgaagaaaaacgcacgcgtccaggagatgatattatctccctgcttcttcaggcaaaagaagaagggaaacagctaacggatgaagagatcgtcggtttttctattttgctactgattgctggaaacgaaacaaccacaaatttaatttcaaatacgatttattgtttaatggaagataaagcttcttttgaacgactcaaacgagagaaagaacttttgccttctgcgattgaagaagttcttcgctatcgttcacccgttcaggctcttcaccgaatcgtaaaagaagatgtgactcttgcaggcaagaaactaaaagcgggcgaacacgtcgttccatggatgggatctgcgcatcgagacgcggagtattttgaagatccggatgtatttaaaatcgatcgaaagccaaatgtgcacatggcatttggaagaggcattcatttttgcttaggagcaccccttgctcgaatagaagcaaaaattatgctggccgagctgattgaccgctatccgcagatggactggagcccgtcatttgagttaaagccaattgaaagcacgtttgtgtacgggttaaaagaattattgattcgtaaaaacgtataa

SEQ ID NO: 11 - DNA, 1212 bp, Bacillus megaterium DSM 32963
atgaatccaaaagcagtgaaaagagaaaatcgttatgctaatttaattcctatgcaagaaattaaatctgttgaacagcagctttacccatttgatatttataatagcctgcgtcaagaggcacctattcgttatgatgaaagccgaaactgctgggatgtatttgattatgaaacagtaaagtatattttaaaaaacccgtcgcttttttcatccaaacgagcaatggaagaacgccaagaaagtattttaatgatggatcctccaaaacatacaaagttacgaaatttagtaaacaaagcttttacgccaagagctattcagcacttggagggtcatattgaagagattgcagattatttattagatgaagtaagcagcaaagaaaagtttgatatagtagaagactttgctggaccactgccgattattgtaatagcagagctgctaggagtaccaatacaagaccgagcactgtttaaaaagtactccgatgacttagtgagcggtgccgaaaataattcagacgaagcttttgcgaaaatgatgcaaaagcgtaatgaaggcgtgatttttttacaagggtattttaaagaaattatcgcggagcgtcagcaaaataaacaagaagatctcatttcgcttcttttagaagcagaaatcgacggcgagcatttaacagaagaggaagtgctggggttttgtattttattgctagtagcaggtaatgagacaacaacaaacttaattacgaacggagttcggtatatgacagaagatgtagacgtgcagaatgaagttcgtcgagacatatcgctagttccgaacctggtagaagaaacgcttcgctattatcctcctattcaagcgattggacgcatagctactgaagatgtagaacttggagagtgtaagattaaaagaggacagcaggtaattagctgggcagcgagtgcaaatagagattcagctaagtttgaatggcctgacacttttgtcgtgcatcgtaaaacaaatcctcacgtgagctttggatttggcatccatttttgcttaggagcaccactagcaagaatggaagggaaaattgcttttactaagttactagaaaaagggggattttccaaagttcagaatcagtctttaaagccaatagatagcccttttgtttttggtgtaaaaaaatatgaaattgcttttaataacgcctga

SEQ ID NO: 12 - DNA, 1233 bp, Bacillus megaterium DSM 32963
atgaacaaagaagtcattcccgttacagaaattccaaaatttcaatcacgtgcagaagagtttttccctatccaatggtataaagaaatgttgaacaactcccccgtttattttcatgaagaaacaaacacatggaatgtattccaatacgaacatgttaaacaagtcttgagcaactacgattttttttcaagcgatggacagagaaccactatttttgttggagataatagtaagaaaaaaagcacatctccaatcaccaaccttacaaacttagatcctcctgaccaccggaaagcgcgatctttgcttgctgcagcctttactcctcgcagtttaaggaactgggaaccgcgaatcaaacaaattgcagcagaacttgtagaagcgatacaaaagaattcaactattaatattgtggacgatttatcttctccttttcctagtctagttatagcagatctattcggcgtaccggtaaaagaccgatatcagtttaaaaaatgggtagacattctgtttcagccttatgatcaagaaagattagaagagattgaacaagaaaagcaacgtgcaggagctgaatattttcaatacctctacccgattgtcattgaaaagcgctccaacctttctgatgatattatctcagacttaattcaagctgaagttgatggtgaaacattcacagataaagaaattgtacacgctaccatgttgcttctaggtgcaggcgttgaaaccacaagtcacgccattgcaaatatgttttattcttttttatatgacgacaagtcgttatacagcgagctgagaaacaatagagaattagctccaaaagcagtagaagaaatgcttcgatatcggtttcatatctccagacgagatcgcacggttaaacaagataacgagctattaggcgttaagctaaaaaaaggagatgtcgttattgcgtggatgagtgcatgtaatatggatgaaactatgtttgaaaaccctttttctgtagacattcatcgtccaactaataaaaagcacttaacattcggaaacggacctcatttttgtttgggcgcccctcttgcccgacttgaaatgaaaattatcttagaagcatttttagaggccttttctcatattgaaccatttgaagactttgaattagaaccgcatttaacagcttcagctacgggtcaatctttaacatacttgcccatgacggtgtacaaataa

SEQ ID NO: 13 - DNA, 1549 bp, Bacillus megaterium strain DSM 33296
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggccacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 14 - DNA, 1549 bp, Bacillus megaterium strain DSM 33296; identical to SEQ ID NO: 13

SEQ ID NO: 15 - DNA, 1188 bp, Bacillus megaterium strain DSM 33296
atgaagaatggatggacaaactttgttattggtaccgtacgtattcgaattgttggaaaaggaattgaacgttttttaaataattgtgtgagacaacagattatgatttcgaatgtccataaagttgacggtcagctagcgacagcaacgatattattgaaagatgtaaaaaaaattcgtatactaattcgcaacgcagattgtaaaatttattttataagaggcagaggttttccttttttaacaaaacgagtaatcaaaaatagtggatttgctttaggttttctttctttttttattattcttggtttgctttccaacatggtgtggaaagtggaaattagcggtgcagaaccacaaacagaacatcaaatgactaagcagcttgccaaaataggtgtaaaaagaggagagtttcaatttctgttagagagtcctgaaaaaattcagcgctatttaacagataatatgaacaatattacctgggtgggagtagaagtgcgcggcacttcttatcacttccaagtggttgaaaaaaatgagcccaagcctcagcaaaaaacaccttaccaacatttaattgctaaaaagaaagccattattacgaatttgtttgtcgaaaaaggacaacctttagtcaaggttaatgattttgtcaacgaaggcgaggtcctagtttccggtattattggaaacgaaaaaaataaaaaagtagtagctgctaaagggaaagtctacggtgaaacatggtataagtctgaggtggaagtgcctttaaaaactgattttcaggtattaacgggtaatggatatacgaagcactatttagattttcaagcttttaaaatgcctctttgggcttttaacaaagaaaaatacgcctcaaaggtgacagaaaaagtggagcatccactctatttttttaaatggaaattacctctgtcgtatgagaaagttgctgtacgagaagaacagaactcgcagcgcgtctattctaaacaagaggcggtggaaaaagctttggaaatcggtcgaaaaaaattactatcaactttaggtgaggatgctaaaataaaaggtgaaaaagttttgcaccaagaacaggacaatggtaaagttagattatcaatacattaccaagttatagaaaatattgcgaacactcaaccaattattcaaggagattga

SEQ ID NO: 16 - DNA, 1923 bp, Bacillus megaterium strain DSM 33296
ttgacgatggaacaaaaagaagtacaagcatatgaagctgatcagatacaagtattagaaggattagaagctgttcgtaaacgtccggggatgtatattggatcgacgagcgcaaagggtttacatcatcttgtatgggaaattgtagataatagtattgatgaagcgctggccggctattgcgatgaaattaatgttattatcgaaaaggataatagtattacagtcaaagataacggtcgtggaattccggttggtattcaagaaaaaatgggcagacctgccgttgaagttatcttaacggttcttcatgccggaggtaaatttggcggcggcggctataaagtatctggtggattacacggtgtaggtgcctcagttgttaacgcactttctacctctttggaagtgcacgtacatcgtgacggtaaagttcattatcaaaaatatgaacgaggtgtaccggctgctgacttaaaagtagttggagaaacagataaaacaggtactgttattcaattccgtccagacagtgaaatttttacagaaacgcttgaatacgattttgacacgttagctaatcgtctgcgtgagttagctttcttaaatcgcggcattaaaattacgattgaagacaaacgtgaagaagataaaagacgtgaatatcactatgaaggcggaattaagtcttacgttgaacacttgaaccgttcgaaagaagtgattcacgaagagcccatctatattgaaggtaatcgagacaacatttctgtagaaattgctattcaatataacgatagctatacaagtaatttatattcttttgcaaacaacattcacacatatgaaggtggaacgcacgaagcaggatttaaaacagcgttaacgcgtgtaattaacgactatgcacgtaaaaacagcgtatttaaagacagtgacgccaatctaacgggtgaagatgttcgtgaaggaattacagctatcatctctattaagcacccagatccgcagttcgaaggacaaacaaaaacaaagctgggaaatagtgaagcaagaacaattactgactctgtgtttgcagaacacttagaaacttacttgctagagaaccctattgtggcgaaaaaggtaattgaaaaaggtttaatggctgcaagagcaagaatggcggctaaaaaagctcgtgagcttacacgccgtaaaagcgcgcttgaaatttcaaacttaccgggtaaattagcagattgttcatcaaaagatccttctattagcgaactttatgtagtagagggtgactctgcaggaggttcagctaagcagggaagaagccgtcatttccaagctattttgcctttacgtggtaaaattatcaacgtagagaaagcgcgtttagataaaattttatctaataacgaaattcgtacaatcattaccgctctaggaacgggtattggtgacgattttgatatttcgaaagcccgctaccataaaattgtgattatgacagatgcagacgtagacggtgcgcatattcgtacgcttcttctaacgttcttctatcgctatatgagacagattattgagcacggatatgtttacattgcccagccgcctctttacaaagttacacagggtaaaaaagtggagtacgcgtacaacgatcgtcaattagaagaggtattagcttctttccctgaaggcgcaaaaccaaaccttcagcgttacaaaggtttaggagagatgaatcctgaacaattatgggaaacaacaatggatccagagttccgtacccttcttcaggtgaacttgcaagatgcaattgaagctgatgagacatttgaaattttaatgggcgacaaagtagaaccacgccgtaatttcattgaagaaaatgctcagtacgtaaaaaatcttgatatttaa

SEQ ID NO: 17 - DNA, 3564 bp, Bacillus megaterium strain DSM 33296
ttgacaggtcaactagttcagtatggacgccaccgccaacgcagaagttatgctcgtattagtgaagttttagagttaccgaacttaatcgagattcaaacggcttcataccaatggtttttagatgaaggtttacgagaaatgttccaagatatttctccaattgatgattttacaggtaacttatcactagaatttattgattacagcttaggtgagccaaagtattcagtaggagagtccaaagaacgtgatgtaacttatgcagcacctcttcgtgttaaggtgcggttaattaataaagaaacaggtgaagtaaaagaccaagatgtgttcatgggagatttcccattgatgaccgaaacaggtacctttgtaattaatggtgctgagcgtgttatcgtatcacagttggttcgttcaccaagtgtttactatagtggaaagcttgataaaaacggtaaaaaaggatatacagcaactgttatccctaaccgtggtgcatggctagaatatgaaactgatgcaaaagatgtagtatacgtgcgtattgatcgtactcgtaaactgccagtaacagtgctgttacgcgcgctaggtttcggttctgaccaagagattatcgatttaatcggtgataatgaatacatccgtaatacgcttgaaaaagataatacggaaacaacggaaaaagcgctattagaaatctatgagcgtttacgtccaggtgaaccaccgacagttgagaatgcgaagtctctattagtatctcgcttctttgatccaaaacgatatgacttagcgaacgtaggtcgctataaaattaataaaaagcttcatatcaaacatcgcttatttaatcaaaagctagctgaaacattagttgatcctgaaacaggtgaaattattgcagaaaaaggcgcaatgattgaccgccgtctgctagatcgcttgattccgatgcttgaaggtggagtaaacttcaaaacttatagtccggttggtggagtagtagaagacgatgttacattacaatctattaagatttatgcgccaaatgatccagaaggtgaaaaaatcatcactgtatcaggtaacgcgtatgtaacagaagaagttaaaaatatcacgcctgctgatattttagcatcaatcagttacttctttaacttgcttcatcaagtaggagacacagatgatatcgaccacttaggtaaccgtcgtctgcgttctgtaggtgaattgttacaaaaccaattccgtatcggtttatctcgtatggaacgtgttgttcgtgaaagaatgtcaattcaagacacgaatacaatcacacctcaacaattaattaatattcgcccagttattgcggcgattaaagagttctttggaagttctcaattatcacagttcatggatcaaacgaatccattaggcgaattgacgcacaaacgtcgtctttcagctctaggacctggtggtttaacgcgtgagcgcgctggttttgaagtgcgtgacgttcactactcccactatggacgtatgtgtccgattgaaacgccagagggtccgaatatcgggttaatcaactcactatcttcttatgcaaaagtaaacaaattcggtttcatcgaaacaccttaccgtcgtatcgatcctgaaacaggtaaagtgacagagcgaattgactacttaacagctgatgaagaagataactatgttgtagcccaagcgaacgctcgtctaggtgatgatggttcattcttagatgaaaatgtcgttgcacgtttccgtggagaaaacacggttatccgtcgcgatcgtttagattatatggatgtatcaccaaaacaagttgtatctgccgctacagcatgtattccattcttagagaacgatgactctaaccgtgcattaatgggtgcgaacatgcaacgtcaagcagtacctttattaaatcctgaagcaccaatcgtaggtacaggtatggaatacgtatctggtaaagactctggtgcagccgtgatttgtaaatatcctggtgttgtagagcgcgtagaagcaaaacaaatttttgttcgccgctatgaagaagtagacggacaaaaagttaaaggtaacttagatcaatacaaattattaaaatttgttcgttctaaccaaggtacttgttacaaccagcgtccaattgtttcagttggcgacgaagtagtaaaaggtgagatcttagccgacggtccttcaatggaaaaaggtgagcttgctttaggacgaaacgtaatggttggtttcatgacatgggatggttacaactatgaggatgccatcatcatgagtgaacgccttgtgaaagacgatgtatatacgtctgttcatattgaagaatatgaatctgagtctcgtgatacgaagcttggacctgaagaaattacgcgtgacattccaaacgtaggtgaagatgcgcttcgtaacttagatgagcgtggaatcatccgcattggtgcagaagtaaaagacggagatcttttagttggtaaagtaacgccaaaaggtgtaacagaactaacagctgaagaacgtcttctacacgctattttcggtgaaaaagcgcgtgaagttcgtgatacttctcttcgtgtaccgcacggcggcggtggaatcattcttgatgttaaagtcttcaaccgtgaagatggcgacgaattaccaccaggtgtaaaccaattagtccgtgtatatattgttcagaagcgtaaaatttctgaaggtgacaaaatggccggtcgtcacggtaacaaaggtgtaatttcacgtattttacctgaagaagatatgccttatctaccagacggtacgccaattgacatcatgttaaacccattaggggtaccatctcgtatgaacatcggtcaggtgctagagcttcatttaggtatggctgctcgtaagcttggcattcacgttgcgtctccagtatttgatggtgcgcgtgaggaagatgtttgggcaacaatcgaagaagctggcatgtctcgtgatgctaaaacagttctatatgatggtcgaacaggtgaaccattcgataaccgtgtatcagtaggaatcatgtacatgatcaaacttgctcacatggtagacgataaacttcacgctcgttctactggaccatactcacttgttacacaacaaccacttggtggtaaagcgcagttcggtggacagcgttttggtgagatggaggtatgggcacttgaagcatacggtgctgcttacacattacaagagatcttaacagtgaaatcagatgacgtagtaggtcgtgtgaaaacatacgaagcaattgttaaaggtgaaaacattccagaacctggcatacctgaatcgttcaaagtattaattaaagaactgcaaagtttaggtatggatgtgaagatgctttctgctgacgagcaagaaattgatatcatggactcagaagaggaccatgagcaaccaacagaatcaattattgctgataacgaagaaagcctttctgaaggacaaaaagatcctgtcacaaaagagtaa

SEQ ID NO: 18 - DNA, 1632 bp, Bacillus megaterium strain DSM 33296
atggcaaaagacattaaatttagcgaagaagcacgtcgcgcaatgctacgtggtgtagatacattagcaaatgctgtaaaagtaacgcttggaccaaaaggtcgtaacgttgtattagaaaagaaattcggttcaccgcttattacaaatgacggtgtaacaattgcaaaagaaatcgaattagaagacgcatttgaaaatatgggtgctaaattagtagccgaagttgctagcaaaacaaacgacgttgctggtgacggtacaactactgcaacagttttagcgcaagcaatgatcagagaaggtcttaaaaacgtaacggctggtgctaacccaatgggtatccgtaaaggtatggaaaaagcagtagctgtagcggttgaagaactaaaagcaatctctaaaccaattcaaggtaaagattcaattgctcaagtagcggctatctcagcagctgacgaagaagtaggtcaattaattgctgaagcaatggagcgcgttggtaacgacggcgttatcacacttgaagaatcaaaaggtttcacaactgaattagaagtggtagaaggtatgcagtttgaccgtgggtatgcatctccttacatggtaactgattcagataaaatggaagctgtattagatgatccatacatcttaatcacagacaaaaaaatcggtaacattcaagaaatcttaccggtattagagcaagttgttcaacaaggcaagcctctattaatcatcgctgaagacgtagaaggcgaagctttagcaacattagttgtgaacaaacttcgtggtacattcacagctgtagctgttaaagctcctggtttcggtgatcgtcgtaaagcaatgctacaagacgttgcgatcttaacaggcggagaagtaatcactgaagagcttggtcttgacttaaaaacagcaagcatcgatcaattaggtcgcgcttctaaaattgtcgtaacaaaagaaaatacaacagttgtaaacggtgcaggaaacgcagaagacatcctagcacgcgtaaaccaaatcaaagctcagcttgaagaaacaacttcagagtttgaccgtgaaaaattacaagagcgcttagcaaaacttgctggtggcgtagctgtaattaaagttggtgcggcaactgaaactgagttaaaagaacgtaaattacgtattgaagatgcattaaactctacgcgtgctgcggttgaagaaggtatcgtagctggtggtggtactgcattagtaaatatctataataaagtagcaagcatcgaagctgatggtgacactgctacaggtatcaacatcgtattacgtgcgattgaagagcctgtacgccaaattgctcacaacgctggtttagaaggatcagtaatcgttgagcgtctaaaaggcgaagctgttggaactggatttaacgctgcaactggcgagtgggtaaatatgctagacactggtatcgttgacccaacaaaagtaacgcgttcagctcttcaaaatgcttcttctgtagcggctatgttcttaacaactgaagcagttgttgctgacaagccagaagaaggcggagcacctgcaatgcctgacatgggcggcatgggtggaatgggcggcatgatgtaa

SEQ ID NO: 19 - DNA, 3150 bp, Bacillus megaterium strain DSM 33296
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggccgtgtaacgcgttacttatcaagtcagcgtctaattaaagaagcatgcgatgaatcacgttttgataaaaacttaagtcaagcgcttaaatttgtacgtgattttgcaggagacgggttatttacaagctggacgcacgaaaaaaattggaaaaaagcgcataatatcttacttccaagcttcagtcagcaggcgatgaaaggctatcatgcgatgatggtcgatatcgccgtgcagcttattcaaaagtgggagcgtctaaatgcagatgagcatattgaagtaccggaagatatgacgcgtttaacgcttgatacaatcggtctttgcggctttaactaccgctttaacagcttttaccgagatcagccgcatccgtttattacgagtatggtccgtgcactggatgaatcgatgaacaagctgcagcgagcaaatccagatgacccagcttatgatgaaaacaagcgccagtttcaagaagatatcaaggtaatgaacaacctagtagataaaattattgcagatcgcaaagcaagcggtgaacaaagcgatgatttgttaacacatatgctaaacggaaaagatccagaaacaggtgagccgcttgatgacgagaacattcgctatcaaattattacattcttaattgcgggacacgaaacaacaagcggtcttttatcatttgcgctgtatttcttagtgaaaaatccacatgtattacaaaaagcagcagaagaagcagcacgagttctagtagatcctgttccaagctacaaacaagtcaaacagcttaaatatgtgggcatggtcttaaacgaagcgctgcgcttatggccaacggctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacgggcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggattcgcaccacaggtcgctacccttgattcgcacaccggaaatcttccccgcgaaggagctgtattaattgtaacggcgtcttataacggacacccgcctgataacgcaaagcaatttgtcgactggttagaccaagcgtctgctgatgaagtaaaaggcgttcgctattccgtatttggatgcggtgataaaaactgggcgacaacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgatgtagcagcctactttaacctcgacattgaaaacagtgaagataataaatctacactttcacttcaattcgtcgacagcgccgcggatatgccgcttgcgaaaatgcacggtgcgttttcagcgaacgtcgtagcaagcaaagaacttcaacagccaggcagtgcacgaagcacgcgtcatcttgaaattgaacttccaaaagaagcttcttatcaagaaggagatcatttaggcgttattcctcgcaactatgaaggaatagtaaaccgtgtaacagcaaggttcggcctagatgcatcacagcaaatccatctggaagcagaagaagaaaaattagctcatttgccactcgctaaaacagtatcagtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatggagttcagcgaatttatcgcccttctgccaagcatacgcccgcgctattactcaatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcaggatttacgctgccaaaagaccctgaaacgccgcttatcatggtcggaccgggaacaggcgtcgcgccgtttagaggtttcgtgcaggctcgcaaacagctaaaagaacaaggacagtcgcttggagaagcgcatttatacttcggctgccgttcacctcatgaagactatctgtaccaagaagagcttgaaaacgcccaaaatgaaggcatcattacgcttcataccgctttttctcgcgtgccaaatcagccgaaaacatacgttcagcacgtgatggaacaagacggcaagaaattgattgaacttcttgatcaaggagcgcacttctatatttgcggagacggaggccaaatggcacctgacgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 20 - DNA, 1812 bp, Bacillus megaterium strain DSM 33296
gtgttgcaacttaaggtagtaaacagcccttttaatcaagaacaagcagatttgcttaatcgccttctgccgacattaacagaagcacaaaaaatgtggttgagcggttatttaacagcatctcaatctacgtctgccgaaggaacgccagacgtttctacagcagcgcctgctcaaacgaagcagacagtttcaaaagacgtaacgattctttacggatcacaaacaggaaatgctcaaggacttgctgaaaatacaggcaaaacgcttgaagcaaaaggctttaatgtaactgtatcttctatgaatgatttcaaaccaaataatttaaagaaacttgaaaatttattaattgtcgtaagtacacatggagaaggagagccgcctgataatgcgctatctttccatgaatttcttcacggccgtcgagcgccaaaacttgagaacttccgtttctctgttttatcgcttggagacagctcatacgaatttttctgtcaaacaggaaaagaatttgatgtgcgcttagcagaacttgggggtgaaagactgtatccgcgcgttgactgtgatttagattttgaagagcccgcaaataaatggcttaaaggtgttattgacggattaagcgaagcgaaaggacacagcgcttcggcagctgttccagcggaagctcctgcaggaacttcaccatattcaagaacaaatccttttaaagcagaagtgcttgagaacttaaacttaaacggccgcggatcaaataaagaaacgcgacacttagaactatctctagaaggttcaggtttgacgtatgaaccaggagacagtttaggtatttatcctgaaaatgacccggagcttgttgatcttcttcttaacgaattcaagtgggatgcaagtgaaagtgtaacggttaataaagaaggagaaacgcgtcctcttagagaagcgctaatctctaattttgaaattaccgttttaacaaagcctcttttaaagcaagcagctgagcttactggaaacgataagttaaaagcgcttgtagaaaatcgcgaggaattaaaagcatacacacaaggccgtgatgtaattgacttagttcgtgacttcggtccatggaacgtatcggcacaagagtttgtagccattctacgtaaaatgccagcgcgtctttactcgattgcaagcagcttatcagcaaaccctgatgaagttcatctaacaatcggagcggtacgctacgaagcgcatggacgggagcgcaaaggtgtttgttcagtcctttgttcagaacgtttgcagccaggcgatacgattcctgtataccttcaaagcaataaaaactttaagcttcctcaagatcaagaaacgccaattattatggtaggacctggtacaggtgtggctccgttccgctcatttatgcaagagcgtgaagaaacaggggcaaaaggcaagtcatggatgttctttggagatcagcacttcgtaacagacttcctttaccaaacagaatggcaaaagtggttaaaagatggcgtactgacaaaaatggacgtggcgttttcacgcgatacagaagaaaaagtatacgtacaaaaccgtatgctcgaacatagtaaagaattattccaatggttagaagaaggcgcatctttttatgtgtgcggagataaaacaaatatggcacgcgacgtgcacaacacgctagttgaaattatcgaaacagaaggcaagatgagccgtgaacaggcggaaggttaccttgctgaaatgaagaaacaaaaacgttatcagcgtgatgtatactga

SEQ ID NO: 21 - DNA, 1278 bp, Bacillus megaterium strain DSM 33296
ttgccggttatacagggcccctcgtcgtataagcttaccggtcacctgcaaaagtttagagagaacccactgggttttttggaaaacttgactcagtatggagaaattgcaacatttagagttgcgcacaaacgcttttatgtaacgagagaccctcaacttattaaagatgtagtcattacaaacagcaaggcgtttcaaaaaattaaactaacgcatatgtttaaaacgctgctaggcgaagaaatgttatggacagatgaagcattatatatgagccccattcaaccctcacaactaaaacaacatctaacttataataaggaagcaatcgcaaaaataattgaaaagcatacggaaacgtgggaagaagggcagcttcgaacgatagtaaaagacataagacagatagtcgtagctgtactccttcaacttgtatttggcatttcgattgaggagaaagataaaattcactatgttcaagcgcttatgagaaaaaaagaaaagttaggcaaaatttatattcgtctacctttgcaccagcttgactcagatgagcagctagaacaactcctttttgaacgcgttcaaatgcgtgttcaaaacaaaaccgaaggaaatgatttactgcagtatatattgaattcttacggagaagacagcgatgaaagagaaatatatgaacagctaaattctatattcctttcgatgtatgaaatgattacgcacgtatgcagctggtctattcatttactgtctcaaaatactagggagcaccttcagctgtataaagaaattcaagcctatgctagcggcgaatcactttcaaccaaaaatctgacctatatgcgcaaaataattgctgaaagcatgaggttgtatccgcctctttggctatttgggcgtcaagcacgtgaagatatacaaatagatggctacagtatcaaaaaaggggagattatgttaatcagcccttatatgatgcatcgccatgaagattattttttagagccaagtgaatttttacctgaccgttttgaaaaaggagggtccattgatgtcccaagctatatgtatatgccgcttgggattgagcatcaagcggaaaggggcattaactatatcactgaaatagtaaccatttttctgtcagaaatgacaaagcgctttttatttcagctgactgagcccgaatctattgctccgatggcgggcgtaatgctgaatatgaaagaagaactgaaggtgaatgttcacaaagttcacgcgcagtcttga

SEQ ID NO: 22 - DNA, 1215 bp, Bacillus megaterium strain DSM 33296
atgaaaaccgaaagagaaaacggaattgtccgtcaagtgaatacaattcaaacaaaagaagagcgctttaatcctttttcatggtatgaagagatgagaaacaccgcgcctgtgcaatgggatgaagaaaggcaggtatgggatgtttttcactatgacggggttaaagaagtgctagaacaaaagaatattttttcttctgatcgaagacctccacaaaaccaaagacaaactgctctaggaacgagtctaattaatattgatccgcctaagcatgctgaaatgagagcacttgttaataaagcttttacgcctaaagcaatgaaagcatgggagcctaaaattgcgcgtattacagctgaattattacaagaagttgagcatcttgaagatattgatatagtcgagcatctgtcctatccgcttccggttatggtaattgccgatattttaggcgtgccgatagaagatcagcgtcagtttaaagattggtccgatattatcgtagccggtccttctaataatgaacgtgaaacgctcgaaaaattgcagcaagagaaaatgaaagcaaatgatgagctagaaacttatttttatcgaatcattgaagaaaaacgcacgcgtccaggagatgatattatctccgtgcttcttcaggcaaaagaagaagggaaacagctaacggatgaagaaatcgtcggtttttctattttgctgctgattgctggaaacgaaacaaccacaaatttaatttcaaatacgatttattgtttaatggaagataaaacttcttttgaacgactcaaacgagagaaagaacttttgccttctgcgattgaagaagttcttcgctatcgttcacccgttcaggctcttcaccgaatcgtaaaagaagatgtgactcttgcaggcaagaaactaaaagcgggcgaacacgtggttccatggatgggatctgcgcatcgagacgcggagtattttgaagacccggatgtatttaaaatcgatcgaaagccaaatgtgcacatggcatttggaagaggcattcatttttgcttaggagcaccccttgctcgaatagaagcaaaaattatgctggccgagctgattgaccgctatccgcagatggactggagcccgtcatttgggttaaacccgattgaaagcacgtttgtgtacgggttaaaagaattattgattcgtaaaaacgtataa

SEQ ID NO: 23 - DNA, 1212 bp, Bacillus megaterium strain DSM 33296
atgaattcaaaagcagtaaaaagagaaaatcgctatgctaacttgattcctatgcaagaaatcaaatcagttgaacagcagctttacccatttgatatttataacagtctgcgtcaaaaggcacctattcgttacgatgaaagccgaaactgctgggatgtatttgattatgaaacagtaaagtatattttaaaaaacccgtcgcttttttcatccaaacgagcaatggaagaacgccaagaaagtattttaatgatggaccctccaaaacatacaaagttacgaaacttagtaaacaaagcttttacgccaagagctattcagcacttagagagtcatattgaagagattgcagattatttattagatgaagtaagcagggaaaaaaagtttgatatagtagaagactttgctggaccactgccgattattgtaatagcagaattgctaggagtaccaatccaagaccgagaactgtttaaaaaatactccgatgacttagtgagcggtgctgaaaataattcagacgaagcttttgcgaaaatgatgcaaaagcgtaatgaaggcgtgatttttttacaagggtattttaaagaaattatcgcggagcgtcagcaaaataaacaagaagatctcatttcgcttcttttagaagcagaaatcgacggcgagcatttaacagaagaggaagtgctggggttttgtattttattgctagtagcaggtaatgagacaacgacaaacttaattacgaacggagtccggtatatgacagaagacgtagacgtgcagaatgaagttcgtcaagacatatcgctagttccgaacctggtagaagaaacgcttcgctattatcctcctattcaagcgattggacgcatagctgctgaagatgtagaacttggagagtgtaagattaaaaaagggcagcaggtaattagctgggcagcaagtgcaaatagagattcagctaagtttgaacggcctgacacttttgtcgtgcatcgtaaaacaaatcctcacgtgagctttggattcggcatccatttttgcttaggagcaccgctagcaagaatggaagggaaaattgcttttactaagttactagagaaaggcgagtttttaaaagttgagaatcagtctttaaagccaataggcagcccttttgtttttggcgtaaaaaaatatgagattacttttaataaagcttaa

SEQ ID NO: 24 - DNA, 1233 bp, Bacillus megaterium strain DSM 33296
atgaacaaagaagtcattcccgttacagaaattccaaaattccaatcacgtgcagaagagtttttccctatccaatggtataaagaaatgctgaataaccaccctgtttattttcatgaagaaacaaacacatggaatgtattccaatacgaccatgttaaacaagtcttgagcaactacgaattcttttcaagcgatgggcaaagaaccactatttttgttggggataatagtaagaaaaaaagcacatctccaattaccaatcttacaaacttagatcctcctgaccaccggaaagcgcgatctttgcttgctgcagcctttactcctcgcagtttaaagaactgggaaccgcgaatcaaacaaattgcagcagaccttgtagaagccatacaaaagaattcaactattaatattgttgacgatttatcttctccttttcctagtttagttatagcagatttattcggtgtaccggtaaaagaccaatatcagttcaaaaaatgggtagatattctgtttcagccttatgaccaagaaagattagaagagattgaacaagaaaagcagcgtgcaggagctgaatattttcaatacctctacccgattgtcattgaaaagcgctccaacctttctgatgatattatctcagacttaattcaagccgaagttgatggtgaaacatttacagatgaagaaattgtacacgctaccatgctgcttttaggtgcaggagttgaaaccacaagtcacgccattgcaaatatgttttattcttttttatatgacgacgagtcgttatacagcgagctgagaaacaatagagaattagctccaaaagcagtagaagaaatgcttcgatatcggtttcacatctccagacgagatcggacggttaaacaagataacgatttattaggcgttaagctaaaaaaaggagatgtcgttattgcgtggatgagtgcatgtaatatggatgagaatatgtttgaaaaccctttttctgtagacattcaccgccaaactaataaaaaacacttaacgtttggaaacggacctcatttttgtttgggcgcccctcttgcccgacttgaaatgaaaatcatattagaagcatttttagaggccttttctcatatcgaaccatttgaagattttgaattagaatcgcatttaacttcttcagctacggggcaatctttaacgtatttgccgatgaaggtgtacagataa

SEQ ID NO: 25 - DNA, 1549 bp, Bacillus megaterium strain DSM 33299
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacgagagtaactgctcgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 26 - DNA, 1549 bp, Bacillus megaterium strain DSM 33299; identical to SEQ ID NO: 25

SEQ ID NO: 27 - DNA, 1188 bp, Bacillus megaterium strain DSM 33299
atgaagaatggatggacaaactttgttattggtaccgtacgtattcgaattgttggaaaaggaattgaacgttttttaaataattgtgtgagacaacagattatgatttcgaatgtccataaagttgacggtcagctagcgacagcaacgatattattgaaagatgtaaagaaaattcgtatactaattcgcaacgcagactgtaaaatttattttataagaggtcgaggttttccttttttaacaaaacgagtgatcaaaaatagtgggtttgctttaggttttctttctttttttattattcttggtttgctttccaacatggtgtggaaagtggaaattagcggtgcagaaccacaaacagaacatcaaatgactaagcagctagccaaaataggtgtaaaaagaggagagtttcaatttctgttagagagtcctgaaaaaattcagcgctatttaacggataatatgaacaatattacctgggtgggagtagaagtgcgcggcacttcttatcacttccaagtggttgaaaaaaatgagcccaagcctcagcaaaaaacaccttaccaacatttaattgctaaaaagaaagccattattacgaatttgtttgtcgaaaaaggacaacctttagtcaaggttaacgattttgtcaatgaaggcgaggtcttagtttccggtattattggaaacgaaaaaaataaaaaagtagtagctgctaaagggaaagtctatggtgaaacatggtataagtctgaggtggaagtacctttaaaaactgattttcaggtattaacgggtaatggatatacgaagcactatttagattttaaagcttttaaaatgcctctttgggcttttaacaaagagaaatacggctcaaaggtgacagaaaaagtggaacatccactctatttttttaaatggaaattacctctgtcgtatgagaaagttgctgtacgagaagaacagaactcgcagcgcgtctattctaaacaagaggcggtggaaaaagctttggaaatcggtcgaaaaaaattactatcaactttaggtgaggatgctaaaataaaaggtgaaaaagttttgcaccaagaacaggacaatggtaaagttagattatcaatacattaccaagttatagaaaatattgcgaacactcaaccaattattcaaggagattga

SEQ ID NO: 28 - DNA, 1923 bp, Bacillus megaterium strain DSM 33299
ttgacgatggaacaaaaagaagtacaagcatatgaagctgatcagatacaagtattagaaggattagaagctgttcgtaaacgtccggggatgtatattggatcgacgagcgctaagggtttacatcatcttgtatgggaaattgtagataatagtattgatgaagcgctggccggctattgcgatgaaattaatgttattatcgaaaaggataatagtattacagtcaaagataacggtcgtggaattccggttggtattcaagaaaaaatgggcagacctgctgttgaagttatcttaacggttcttcatgccggaggtaaatttggcggcggcggctataaagtatctggtggattacacggtgtaggtgcctcagttgttaacgcactttctacctctttggaagtacacgtacatcgtgatggtaaagttcattatcaaaaatatgaacgaggtgtaccagctgctgacttgaaagtagtcggagaaacagataaaacaggtactgttattcaattccatccagacggtgaaatttttacagaaacgcttgaatacgattttgatacgttagctaatcgtctgcgtgagttagctttcttaaatcgcggcattaaaattacgattgaagacaaacgtgaagaagataaaagacgtgaatatcactatgaaggcggaattaagtcttacgttgaacacttaaaccgttcgaaagaagtgattcacgaagagccaatctatatcgaaggtaatcgagacaacatttctgtagaaattgctattcaatataacgatagctatacaagtaatttatattcttttgcaaacaacattcacacatatgaaggtggaacgcacgaagcaggatttaaaacagcgttaacgcgtgtaattaacgactatgcacgtaaaaacagcgtatttaaagacagtgacgccaatttaacgggtgaagatgttcgtgaaggaattacagctatcatctctattaagcacccagatccgcagttcgaaggacaaacgaaaacaaagctgggaaatagtgaagcaagaacaattactgactctgtgtttgcagaacacttagaaacttacttgctagagaaccctattgtggcgaaaaaggtaattgaaaaaggtttaatggctgcaagagcaagaatggcagctaaaaaagctcgtgagcttacaagacgtaaaagcgcgcttgaaatttcaaacttaccgggtaaattagcagattgttcatcaaaagatccttctattagcgaactctatgtagtagagggtgactctgccggcggttcagctaagcagggaagaagccgtcatttccaagctattttgcctttgcgcggtaaaattatcaacgtagagaaagcgcgtttagataaaattttatctaataacgaaattcgtacaatcattaccgctctaggaacgggtattggtgacgattttgatatttcgaaagcccgctaccataaaattgtgattatgacagatgcagacgtagacggtgcgcatattcgtacgcttcttctaacgttcttctatcgctatatgagacagattattgagcacggatatgtgtacattgcccagccgcctctttacaaagtttcacaaggtaaaaaagtggagtacgcgtacaacgatcgtcaattagaagaggtattagcttctttccctgaaggcgcaaaaccaaaccttcagcgttacaaaggtttaggagagatgaatcctgaacaattatgggaaacaacaatggatccagaattccgtactcttcttcaggtgaacttgcaggatgcaattgaagctgatgagacatttgaaattttaatgggcgacaaagtagaaccacgccgtaatttcattgaagaaaatgctcagtacgtaaaaaatctcgatatttaa

SEQ ID NO: 29 - DNA, 3564 bp, Bacillus megaterium strain DSM 33299
ttgacaggtcaactagttcagtatggacgccaccgccaacgcagaagttatgctcgtattagtgaagttttagagttaccgaacttaatcgagattcaaacggcttcataccaatggtttttagatgaaggtttacgagaaatgttccaagatatttctccaattgatgattttacaggtaacttatcactagaatttattgattacagcttaggtgagccaaagtattcagtaggagagtctaaagaacgtgatgtaacttatgcagcacctcttcgtgttaaggtgcggttaattaataaagaaacaggtgaagtaaaagaccaagatgtgttcatgggagatttcccattgatgaccgaaacaggtacctttgtaattaatggtgctgagcgtgttattgtatcacagttggttcgttcaccaagtgtttactatagtggaaagcttgataaaaacggtaaaaaaggatatacagcaactgttatccctaaccgtggtgcatggctagaatatgaaactgatgcaaaagatgtagtatacgtgcgtattgatcgtactcgtaaactgccagtaacagtgctgttacgcgcgctaggtttcggttctgaccaagagattatcgatttaatcggtgataatgaatacatccgtaatacgcttgaaaaagataacacggaaacaacggaaaaagcgctattagaaatctatgagcgtttacgtccgggtgaaccaccgacagttgagaatgcgaaatctctattagtatctcgcttctttgatccaaaacgatatgacttagcgaacgtaggtcgctataaaattaataaaaagcttcatatcaaacatcgcttatttaatcaaaaactagctgaaacattagttgatcctgaaacaggtgaaattattgcagaaaaaggcgcaatgattgaccgacgtctgctagatcgcttgattccgatgcttgaaggtggagtaaacttcaaaacttatagtccagttggtggagtagtagaagacgatgttacattacaatctattaagatttatgcgccaaatgatccagaaggtgaaaaagtcatcactgtatcaggtaacgcatatgtaacagaagaagttaaaaatatcacacctgctgatattttagcatcaatcagttacttctttaacttgcttcatcaagtaggagacacagatgatatcgaccacttaggtaaccgtcgtctgcgttctgtaggtgaattgttacaaaaccaattccgtatcggtttatctcgtatggaacgtgttgttcgtgaaagaatgtcaattcaagacacgaatacaatcacacctcaacaattaattaatattcgtccagttattgcggcgattaaagagttctttggaagttctcaattatcacagttcatggatcaaacgaatccattaggcgaattgacgcacaaacgtcgtctttcagctctaggacctggtggtttaacgcgtgagcgcgctggtttcgaagtgcgtgacgttcactactcccactatggccgtatgtgtccgattgaaacgccagaggggccgaatatcgggttaatcaactcactttcttcttatgcaaaagtaaacaaattcggtttcatcgaaacaccttaccgtcgtatcgatcctgaaacaggtaaagtgacagagcgaattgactacttaacagctgatgaagaagataactatgttgtagcccaagcgaacgctcgtctaggtgatgatggttcattcttagatgaaaatgtcgttgcacgtttccgtggagaaaacacggttatccgtcgcgatcgtttagactatatggatgtatcaccaaaacaagttgtatctgccgctacggcatgtatcccattcttagagaacgatgactctaaccgtgcattaatgggtgcgaacatgcaacgtcaagcagtacctttattaaatcctgaagcaccaatcgtaggtacaggtatggaatatgtatctggtaaagactctggtgcagccgtgatttgtaaataccctggcgttgtagagcgcgtagaagcaaaacaaatttttgttcgccgctatgaagaagtagacggacaaaaggttaaaggcaacttagatcaatacaaattattaaaattcgttcgttctaaccaaggtacttgttacaaccagcgtccaattgtttcagttggcgacgaagtagtaaaaggtgagatcttagccgatggtccttcaatggaaaaaggtgagcttgctttaggacgaaacgtaatggttggtttcatgacatgggatggttacaactatgaggatgccatcatcatgagtgaacgccttgtgaaagacgatgtatatacgtctgttcatattgaagaatatgaatctgagtctcgtgatacgaaacttggacctgaagaaattacgcgtgacattccaaacgtaggtgaagatgcgcttcgcaacttagatgagcgtggaatcatccgcattggtgcagaagtaaaagatggagatcttttagttggtaaagtaacgccaaaaggtgtaacagaactaacagctgaagaacgtcttctacacgctattttcggtgaaaaagcgcgtgaagttcgtgatacttctcttcgtgtaccgcacggcggcggtggaattattcttgatgttaaagtcttcaaccgtgaagatggcgacgaattaccaccaggtgtaaaccaattagtccgtgtatatattgttcagaagcgtaaaatttctgaaggtgacaaaatggccggtcgtcacggtaacaaaggtgtaatttcacgtattttaccggaagaagatatgccttacctaccagacggtacgccaattgacatcatgttaaacccattaggggtaccatctcgtatgaacatcggtcaggtgctagagcttcatttaggtatggctgctcgtaagcttggcattcacgttgcgtctccagtatttgatggtgcgcgtgaggaagatgtttgggcaacaatcgaagaagctggcatgtctcgtgatgctaaaacagttctatatgatggtcgaacaggtgaaccattcgataaccgtgtatcagtaggaatcatgtacatgatcaaacttgctcacatggtagacgataaacttcacgctcgttctactggaccatactcacttgttacacaacaaccacttggtggtaaagcgcagttcggtggacagcgttttggtgagatggaggtatgggcacttgaagcatacggtgctgcttacacattacaagagatcttaacagtgaaatcagacgacgtagtaggtcgtgtgaaaacatacgaagcaattgttaaaggtgaaaacattccagaacctggcatacctgaatcgttcaaagtattaattaaagaactacaaagtttaggtatggatgtgaagatgctttctgctgacgagcaagaaattgatatcatggactcagaagaggaccatgagcaaccaacagaatcaattattgcagataacgaagaaagcctttctgaaggacaaaaagatcctgtcacaaaagagtaa

SEQ ID NO: 30 - DNA, 1632 bp, Bacillus megaterium strain DSM 33299
atggcaaaagacattaaatttagcgaagaagcacgtcgcgcaatgctacgtggtgtagatacattagcaaatgctgtaaaagtaacgcttggaccaaaaggtcgtaacgttgtattagaaaagaaattcggttcaccgcttattacaaatgatggtgtaacaattgcaaaagaaatcgaattagaagacgcatttgaaaacatgggtgctaaattagtagccgaggttgctagcaaaacaaacgacgttgctggtgacggtacaactactgcaacagttttagcgcaagcaatgatcagagaaggtcttaaaaacgtaacggctggtgctaacccaatgggtatccgtaaaggtatggaaaaagcagtagctgtagcggttgaagaactaaaagcaatctctaaaccaattcaaggtaaagattcaattgctcaagtagcggctatctcagcagctgacgaagaagtaggtcaattaattgctgaagcaatggagcgcgttggtaacgacggcgttatcacacttgaagaatcaaaaggtttcacaactgaattagaagtggtagaaggtatgcagtttgaccgtggatatgcatctccttatatggtaactgattcagataaaatggaagctgtattagatgatccatacatcttaatcacagacaaaaaaatcggtaacattcaagaaatcttaccggtattagagcaagttgttcaacaaggcaagcctctattaatcatcgctgaagacgtagaaggcgaagctttagcaacattagttgtgaacaaacttcgtggtacatttacagctgtagctgttaaagctcctggttttggtgatcgtcgtaaagcaatgctacaagacgttgcgatcttaacaggcggagaagtaatcactgaagagcttggtcttgacttaaaaacagcaggcatcgatcaattaggtcgcgcttctaaaattgttgtaacaaaagaaaatacaacagttgtaaacggtgcaggaaacgcagaagatatcctagcacgcgtaaaccaaatcaaagctcagcttgaagaaacaacttcagagtttgaccgtgaaaaattacaagagcgcttagcaaaacttgctggtggcgtagctgtaattaaagtgggtgcggcaactgaaactgagttaaaagaacgtaaattacgtattgaagatgcattaaactctacgcgtgctgcggttgaagaaggtatcgtagctggtggtggtactgcattagtaaatatctataataaagtagcaagcatcgaagctgacggtgacactgctacaggtatcaacatcgtattacgtgcgatcgaagagcctgtacgtcaaatcgctcacaatgctggtttagaaggatcagtaatcgttgagcgtctaaaaggcgaagcagttggaactggattcaatgctgcaactggcgagtgggtaaacatgctagacactggtatcgttgacccaacaaaagtaacgcgttcagctcttcaaaatgcttcttctgtagcggctatgttcttaacaactgaagcagttgttgctgacaagccagaagaaggcggagcacctgcaatgcctgacatgggcggcatgggtggaatgggcggcatgatgtaa

SEQ ID NO: 31 - DNA, 3150 bp, Bacillus megaterium strain DSM 33299
atgacaattaaagaaatgcctcagccaaaaacgtttggagagcttaaaaacttaccgttattgaacacagacaaaccacttcaaacgctgatgaaaattgcggatgaattaggggaaatctttaaatttgaagcgcctggccgtgtaacgcgctacttatcaagtcagcgtctaattaaagaagcatgtgatgagtcccgattcgataaaaacttaagtcaagctcttaaatttgtgcgtgattttgcaggagacggtttatttacaagctggactcatgagaaaaactggaaaaaagcacacaacatcttacttccaagctttagccagcaggcaatgaaaggctatcacgcaatgatggtcgatatcgccgtgcagcttattcaaaaatgggagcgtctaaatgcagatgagcatattgaagtaccggaagacatgacgcgcttaacgcttgacacaatcggtctttgcggctttaattaccgctttaacagcttttatcgcgatcagccgcacccgtttattacaagtatggtccgtgcactggatgaatcgatgaaaaagctgcagcgtgcaaatccggatgacccagcttatgatgaaaataagcgccagtttcaagaagatgttaaggtgatgaacaacctagtagataaaattattgcagatcgcaaagcaagcggtgaacaaagtgatgatctattaacgcatatgctaaacggaaaagatccagaaacaggtgagccgcttgatgacgagaacattcgctatcaaattattacgttcttaattgcgggacacgaaacaacaagcggtctgttatcatttgcgctgtatttcttagtgaaaaatccacatgtattacaaaaagcagcagcagaagcagcacgagttctagtagatcctgttccaacctacaaacaagtcaaacagcttaaatatgtgggcatggtcttaaatgaagcgctgcgcttatggccgacggctcctgcgttttctctatatgcaaaagaggatacggtgcttggaggagaatatccgttagaaaaaggggatgaactgatggttctgattcctcagcttcaccgtgataaaacgatttggggagacgatgtagaagagttccgcccagagcgttttgaaaacccaagcgcgattccgcagcatgcgtttaaaccatttggaaacggtcagcgcgcgtgtatcggtcagcagttcgctcttcatgaagcaacgcttgtacttggtatgatgctaaaacactttgactttgaagatcatacaaactacgagctagatattaaagaaacgttaacgttaaaacctgagggctttgtggtaaaagcaaaatcgaaacaaattccgcttggcggcattccttcaccaagccgagagcagtcagctaaaaaagagcgcaaaaccgtagaaaacgctcataatacgccgctgcttttgctatacggttcaaatatgggaacagccgaaggaacggcgcgggatttagcggatattgcgatgagcaaaggattcgcaccgcaagtcgcaacgcttgattcccacgcaggaaatcttccgcgtgaaggagctgtgttaattgtaacagcttcgtataacggacatccgcctgataacgcaaagcagtttgtcgagtggttagaccaagcgtctgctgatgaagtcaaaggcgttcgctactccgtatttggatgcggcgataaaaactgggctactacgtatcaaaaagtgcctgcttttattgatgaaacgcttgcggctaaaggagcagaaaacatcgctgaacgcggcgaagcagatgcaagcgacgactttgaaggcacatacgaagaatggcgtgaacacatgtggagtgacgtagcagcctactttaacctcgacattgaaaacagtgaagataatcaatctacgctttcacttcaatttgtcgacagcgctgcggacatgccgcttgcgaaaatgcacggtgcgttttcagcaaacgtcgtagcgagcaaagaacttcaacagccaggcagtgcacgaagcacgcgacaccttgaaattgaacttccaaaagaagcttcttatcaagaaggagatcatttaggtattattcctcgcaactatgaaggaatagtaaatcgtgtaacagcaaggttcgatgtagatgcatcacagcaaatccgtctggaagctgaagaagaaaaattagcgcatttgccactcggtaaaacagtatcagtagaagagcttctacaatatgtagagcttcaagatcctgttacgcgcacgcagcttcgcgcaatggctgctaaaacggtctgcccgccgcataaagtggagcttgaagccttgcttgaaaagcaagcgtacaaagaacaagtactggcaaaacgcttaacaatgcttgaactgcttgaaaaatacccggcgtgtgaaatggaattcagcgaatttatcgctcttcttccaagcatgcgtccgcgctattactcaatttcttcatcaccttgcgtcgatgaaaaacaagcaagcatcacggtcagcgttgtttcaggagaagcgtggagcggatatggagaatacaaaggaattgcgtcgaactatcttgccgagctgcaagaaggagatacgattacgtgctttgtttccacaccgcagtcaggctttacgctgccaaaagaatctgaaacacctattatcatggtcggaccgggaacaggcgtcgcgccatttagaggctttgtgcaggctcgcaagcagttaaaagaacaaggacagttgcttggagaagcgcatttatactttggctgccgttcacctcatgaagactatctgtatcaagaagagcttgaaaacgcacaaaatgaaggtgtcattacgcttcataccgctttttcacgcgtaccaaatcagccgaaaacatacgttcagcgcctgatggaacaagacggtatgaaattgattgaacttcttgatcaaggagcgcatttctacatttgcggagacggaagccaaatggcacctgacgttgaagcaacgcttattaaaagctatgctgatgttcataaagtaagtgaagcagacgctcgcttatggctgcagcagctagaagaaaagggccgatacgcaaaagacgtgtgggctgggtaa

SEQ ID NO: 32 - DNA, 1812 bp, Bacillus megaterium strain DSM 33299
gtgttgcaacttaaggtagtaaacagcccttttaatcaagaacaagcagatttgcttaatcgccttctgccgacattaacagaagcacaaaaaatgtggttgagtggttatttaacagcagctcaatctacgtctgccgaaggaacgccagccgtttctacagcagcgcctgctcaaacgaagcagacagtttcaaaagatgtaacgattctttacggatcacaaacaggaaatgctcaaggacttgctgaaaatacaggcaaaacgcttgaagcaaaaggttttaatgtaactgtatcttctatgaatgatttcaaaccaaataatttaaagaaacttgaaaatttattaattgttgtaagtacacatggagaaggagagccgcctgataacgccctgtctttccacgaatttcttcacggccgtcgagcgccaaaacttgagaatttccgtttttctgtcttgtcgcttggagacagctcatacgaatttttctgtcaaaccggaaaagaatttgatgtgcgcttagcagaacttggcggtgaaagactgtatccgcgcgttgactgtgatttagattttgaagaacccgcaaataaatggcttaaaggtgttattgacggattaagcgaagcgaaaggacacagcgcttcggcagctgttccggcggaagctcctgcaggaacttcaccatattcaagaacaaatccttttaaagcagaagtgcttgagaacctaaacttaaacggccgcggatcaaataaagaaacgcgacacttagaactatctctagaaggttcaggcttgacgtatgaaccaggagacagtttaggtatttatcctgaaaatgacccggagcttgttgatcttcttcttaacgaattcaagtgggatgcaagtgaaagtgtaacggttaataaagaaggagaaacgcgtcctcttagagaagcgctaatctctaattttgaaattaccgttttaacaaaaccgcttttaaagcaagcagctgagcttactggaaacgataagttaaaagcgcttgtagaaaatcgcgaggaattaaaagcatacacacaaggccgtgatgtgattgacttagttcgtgacttcggcccatggaacgtatcggcacaagagtttgtagccattttacgtaaaatgccagcgcgcctttactcgattgcaagcagtttatcagcaaaccctgatgaagttcatctaacaatcggagcggtacgctacgaagcgcatggacgcgagcgtaaaggtgtttgttcagtcctatgttcagaacgtttgcagccaggcgatacgattcctgtataccttcaaagcaataaaaactttaagcttcctcaagatcaagaaacgccaattattatggtaggacctggtacaggtgtggctccgttccgctcatttatgcaagagcgtgaagaaacaggagcaaaaggcaagtcatggatgttctttggagatcagcacttcgtaacagacttcctttaccaaacagaatggcaaaagtggttaaaagatggcgtactgacaaaaatggacgtggcgttttcacgcgatacagaagaaaaagtatacgtacaaaaccgtatgctcgaacatagcaaagaattattccagtggttagaagaaggcgcatctttttatgtgtgcggagataaaacaaatatggcacgcgacgtgcacaacacgctagttgaaattatcgaaacagaaggcaagatgagccgtgaacaggcggaaggttaccttgctgaaatgaagaaacaaaaacgttatcagcgtgatgtatactga

SEQ ID NO: 33 - DNA, 1278 bp, Bacillus megaterium strain DSM 33299
ttgccggttatacagggcccctcgtcgtataagcttaccggtcacctgcaaaagtttagagagaacccactgggttttttggaaaacttgactcagtatggagaaattgcaacatttagagttgcgcacaaacgcttttatgtaacgagagaccctcaacttattaaagatgtagtcattacaaacagcaaggcgtttcaaaaaattaaactaacgcatatgtttaaaacgctgctaggtgaagaaatgttatggacagatgaagcattatacatgagccccattcaaccctcacaacttaaacaacatctaacttataataaggaagcaatcgcaaaaataattgaaaagcatatggaaacgtgggaagaagggcagcttcgaacgatagtaaaagatattagacaaatagtcgtagctgtactccttcaacttgtatttggcatttcgattgaggataaagataaaattcactatgttcaagcgcttatgagaaaaaaagaaaagttaggcaaaatttatattcgtctacctttgcaccagcctgactcagatgagcagctagaacaactcctttttgaacgcgttcaaatgcgtgttcaaaacaaaaccgaagaaaatgatttactgcagtatgtattacattctcacggggaagacatcgatgaaagagaaatatatgaacagctaaattctatattcctttcgatgtatgaaatgattacgcacgtatgcagctggtctattcatttactgtctcaaaatactagggagcaccttcagctgcataaagaaattcaagcctatgctagcggtgaatcactttcaaccaaaaatctgacctatatgcgcaaaataattgctgaaagcatgaggctgtatccgcctctttggctatttgggcgtcaagcacgtgaagatatacaaatagatggttacagcattaaaaaaggggagattatgttaattagcccttatatgatgcatcgccatgaagattattttttagagccaagtgaatttttacctgaccgttttgaaaaaggagggtccattgatgtcccaagctatatgtatatgccgcttgggattgagcatcaagcggaaagaggcatggactatatcactgaaatagtaactatttttctgtcagaaatgacaaagcgctttttatttcaactgactaaggccgagtctattgctccgatggcgggcgttatgctgaatatgaaagaagaactgaatgtgaatgttcacaaagttcacacgcagtcttga

SEQ ID NO: 34 - DNA, 1215 bp, Bacillus megaterium strain DSM 33299
atgaaaaccgaaagagaaaacggaatcgtccgtcaagtgaatacgattcaaacaaaagaagagcgctttaatccttttacatggtatgaagagatgagaaacagcgcgcctgtgcaatgggatgaagaaaggcaggtatgggatgtttttcactatgacggggtcaaagaagtgctggaacaaaagaatattttttcttctgatcgaagacctccacaaaaccaaagacaaactgctctaggaacgagcctaattaatattgatccgcctaagcatgctgaaatgagagcgcttgttaataaagcttttacgcctaaagcaatgaaagcatgggagcctaaaattgcgcgtattaccgctgaattattacaagaagttgagcatcttgaagatattgatatagtcgagcatctttcctatccgcttccggttatggtaattgccgatattttaggcgtgccgatcgaagatcagcgtcagtttaaagattggtcggatattatcgtagcgggtccatcgaataatgaacgtgaaacgctcgaaaaattgcagcaagagaaaatgaaagcaaacgatgagttagaaacttacttttatcgaatcattgaagaaaaacggacgcagccaggagatgatattatttccgtgcttcttcaagcaaaagaagaagggaagcagctaacggatgaagaaatcgtcgggttttccattttgctgctgattgcaggaaacgaaacaactacgaacttaatttcaaatacaatttattgtttaatggaagataaggcttcttttgaacacctcagacgtgagaaagagcttttgccttctgcgatcgaagaagttcttcgctatcgttcacccgttcaggctcttcaccgaatcgtaaaagaagatgtgactcttgccggtcagaaactaaaagcgggcgaacacgtcgttccatggatgggatctgcgcatcgagatgctcagtactttgaagacccggatgtatttaaaatcgatcgaaagccaaatgtgcacatggcatttggaagaggcattcatttttgcttaggagcaccgcttgctcgaatagaagcaaaaattatgctggctgagctgattgaccgctatccgcagatggactggagcccagcatttgagttgaagccgattgaaagcacgtttgtgtacgggttaaaagaattattaattcgtaaacatgtataa

SEQ ID NO: 35 - DNA, 1212 bp, Bacillus megaterium strain DSM 33299
atgaacccaaaagcagtgaaaagagaaaatcgttatgctaacttaattcctatgcaagaaattaaatccgttgaacagcagctttacccatttgatatttataacagtttgcgtcaagaagcacctattcgttacgatgaaagccgaaactgctgggatgtatttgattatgaaacagtaaagtatattttaaaaaacccgtcgcttttttcatccaaacgagcaatgaaagaacgccaagaaagtatcttaatgatggatcctccaaaacatacaaaattacgaaacttagtgaataaagcttttacaccaagagctattcagcacttggagggtcatattgaagagattgcagattatttattagatgaagtaagcagcaaaaaaaagtttgatatagtagaggattttgctggaccactgccgattattgtaatagcagagctactgggcgtacccatacaagaccgagcactgtttaaaaagtactccgatgacttagtgagcggttctgaaaataattcagacgaagcctttgcgaaaatgatgcaaaagcgtaatgacggtgtgctttttttacaagggtattttaaagaaattatcgctaagcgtcagaaagataaacaagaagatcttatttcgctgcttttagaagcagaaattgacggcgagcatttaacagaagaggaagtactggggttttgtattctattgctagtagcaggcaatgaaacaacaacaaacttaattacgaacggagtccggtatatgacagaagatgtagacgtgcagaatgaagttcgtcaagacatatcgctagttccgaatctcgtggaagaaacgcttcgttattatcctcctattcaagcgattggacgcatagccgctgaagatgtagaacttggagagtgtaaaattaaaaaagggcagcaggtaattagctgggcagcaagtgcaaatagagattcagctaagtttgaacggtctaacacttttgtcgtacatcgtaaagcgaatcctcacgtaagctttggatttggcattcatttttgcttaggagcaccgctagcaagaatggaagggaaaattgcctttgctaaattactagaaaaaggggaattttccaaagttcagaatcagtctttaaagccaatagacagcccttttgtttttggtgtaaaaaaatacgaaattacttttaataacgtctga

SEQ ID NO: 36 - DNA, 1215 bp, Bacillus megaterium strain DSM 33299
atggctgtcaaaaaagtgattaacttagctccaataacgcccatgaaagaattaaatacaaaagagaagtattttaatccgtttccaatctatgaaatgtttcgagaagaaaatccggtacgctatgatgaggaaagaaagtgctgggatatctttttatatgaagatgtagactacgttcttaaacatcacgagctcttttcttctcagcagcagcgaatggtagaaggggaaaacttattaagaatggatcctccccgtcataaacaaatgcgcgatctcgttaataaagcgtttacgcctagagaaattaataatcttgctcctcgtattgaagaaattactaatcacttgctgcaaaatgtcatagacaaaggagaaatgaaaatgattcatgactttgccatgccgcttccggtcatcgtcatagctgatttgttaggcgttccggccgaggacagggaaaagtttaaggagtggtctgatatccttgtaaaaggagtaagtgaaaataagcctgaagcgtacgaagcagtgaagcaggaacagcagattgcccgcgatgagttaaaggaatatcttcatagaattgtgcatataagacgtttcgatcctaaacaagatatcatatcagctttactttcatcagaagttgacggaggtaagcttgcagacagcgaaatattagagttcagtatgcttctcttggctgctggtaatgaaacgacgacaaacttaattacaaatggagtccgtctgcttactgaaataccgagtctgcaaaaacaagtgaaaaatgatctttcacttgtgccaactatggtggaggaggttctccggttttatccacctgtacaggcgccttcacgtattgccataaaagacgttgaacttagaggtaagacaataagaaaaggggatgcggtgacagcctgggtagcatcagctaaccgagatgaagctaaatttcctcatgctaatgtatttcaagcggatcgtaagcccaatcagcatcttacttttgggaaaggaattcatttctgcttaggtgctcccttagcccgtctagaagcaaaaattgcgtttgaagaaatgctaagtaaatgtacgaatcttcaattaaaagaaggttcattaatagaacgaactccggcaccgcaaatgtacggtgtggtagaatatcctgttacattttcagtgagcccttcattatag

SEQ ID NO: 37 - DNA, 1233 bp, Bacillus megaterium strain DSM 33299
atgaacaaagaagtcattcccgtgacagaaattccaaaatttcaatcacgtgcagaagagtttttccctatccaatggtataaagaaatgttaaacaattcccccgtttattttcatgaagaaacaaacacatggaatgtattccaatacgaacatgttaaacaagtcttgagcaactacgaatttttttcaagcgatggacaaagaaccactatttttgttggagataatagcaagaaaaaaagcacatctccaatcaccaaccttacaaacttagatcctcctgaccaccggaaagcgcgatctttgcttgctgccgcctttactcctcgcagtttaaagaactgggagccgcgaatcaaacaaattgcagcagaccttgtagaagagacacaaaagaattcaactattaatattgttgaagatttgtcttctccttttcctagtttagttatagcagatttattcggcgtaccggtaaaagaccgatatcagttcaaaaagtgggtagatattctttttcagccttatgaccaggaaagattaaaagagattgaacaagaaaagcaacgtgcaggagctgaatattttcaatatctctacccgattgtcgttgaaaagcgctccaacctttctgatgatattatttcagacttaattcaagcggaagttgatggggaaacattcacagatgaagaaattgtgcacgctaccatgctgcttttaggtgcaggagttgaaaccacaagtcacgccattgcaaatatgttttattctttcttatatgacgacaagtcgttatacagcgagctgagaagcaatagagaattagctccaaaagcagtagaagaaatgcttcgatatcggtttcatatctccagacgagatcgcacggttaaacaagataacgagctattaggcgttaagctaaaaaaaggagatgtcgtgattgcgtggatgagtgcatgtaatatggatgaaaatatgtttgaaaaccctttttctgtagacattcatcgtccaactaataaaaagcacttaacattcggaaacggacctcatttttgtttgggcgcccctcttgcccgacttgaaatgacaattatcttagaagcatttttagaggccttttctcatatcgaaccatttgaaggttttgaattagaaccgcatttaacaccttcagctacgggtcaatctttaacatacttgcccatgacggtgtacaaataa

(19) United States
(12) Patent Application Publication
(10) Pub. No.: US 2021/0392935 A1
(43) Pub. Date: Dec. 23, 2021

(54) PREPARATION COMPRISING A PROBIOTIC STRAIN OF THE GENUS BACILLUS MEGATERIUM AND A POLYUNSATURATED FATTY ACID COMPONENT

(71) Applicant: Evonik Operations GmbH, Essen (DE)

(72) Inventors: Bodo SPECKMANN, Kahl (DE); Ines OCHROMBEL, Essen (DE); Martin SCHILLING, Bonn (DE); Mario GOMEZ, Darmstadt (DE); Michael SCHWARM, Alzenau (DE); Stefan PELZER, Gütersloh (DE); Jessica KLEINBÖLTING, Bielefeld (DE); Thomas BERNGRUBER, Hamm (DE)

(73) Assignee: Evonik Operations GmbH, Essen (DE)

(21) Appl. No.: 17/296,465

(22) PCT Filed: Nov. 28, 2019
(86) PCT No.: PCT/EP2019/082924

§ 371 Date: May 24, 2021

(30) Foreign Application Priority Data
Nov. 30, 2018 (EP) 18209509.1

(51) Int. Cl. A23L 33/12; A23L 33/135; A23L 33/17; A61K 31/202

(52) U.S. Cl. CPC A23L 33/12; A61K 31/202; A23L 33/17; A23L 33/135

(57) ABSTRACT

The invention relates to preparation comprising at least one probiotic strain belonging to the genus Bacillus megaterium, and a polyunsaturated fatty acid component comprising at least one omega-3 or omega-6 fatty acid selected from eicosapentaenoic acid (EPA), docosahexaenoic acid (DHA), arachidonic acid (ARA), alpha linolenic acid, stearidonic acid, eicosatetraenoic acid, docosapentaenoic acid, linoleic acid, γ-linolenic acid, wherein the polyunsaturated fatty acid component comprises an omega-3 or omega-6 fatty acid salt. The invention further relates to use of the inventive preparation as a feed or food supplement, pharmaceutical product, feed-or foodstuff compositions and for topical applications.

The current invention concerns a preparation comprising at least one probiotic strain belonging to the genus Bacillus megaterium or an extract of Bacillus megaterium, and a polyunsaturated fatty acid component comprising at least one omega-3 or omega-6 fatty acid selected from eicosapentaenoic acid (EPA), docosahexaenoic acid (DHA), arachidonic acid (ARA), alpha linolenic acid, stearidonic acid, eicosatetraenoic acid, docosapentaenoic acid, linoleic acid, γ-linolenic acid and/or derivatives thereof.

Dietary intake of omega-3 fatty acids, namely alpha-linoleic acid (ALA), EPA and DHA, is beneficial for human health, in particular with respect to e.g. the amelioration of rheumatoid arthritis and reduction of cardiovascular disease risk factors [1, 2]. Various seafood products are a source of dietary EPA/DHA, but their consumption is often not sufficient to meet the recommended dietary allowance (typically 500 mg EPA and DHA per day) [3]. This gap is closed by the widespread use of dietary supplements or fortified foods containing omega-3 fatty acids [4]. Dietary supplements are concentrated sources of nutrients or other substances with a nutritional or physiological effect, whose purpose is to supplement the normal diet (www.efsa.europa.eu/en/topics/topic/food-supplements). For example, omega-3 fatty acid supplements often contain either triglycerides or omega-3 ethyl esters of EPA/DHA from fish oil, krill oil, or algae.

Omega-3 fatty acids in general have anti-inflammatory, cardio- and neuroprotective effects [2, 5]. Their modes of action involve e.g. direct scavenging of reactive oxygen species, alteration of cell membrane fluidity, which subsequently affects cellular signaling events, modulation of the activity of transcription factors such as PPARG and NFKappaB that orchestrate the biosynthesis of pro-and anti-inflammatory cytokines, and competitive exclusion of substrates that are converted to proinflammatory cytokines by cyclooxygenases and lipoxygenases.

More recently, several oxygenation products of omega-3 and omega-6 fatty acids have been identified and functionally characterized as crucial mediators of their beneficial health effects, in particular with respect to the amelioration of chronic inflammatory conditions [6]. These products include maresins (MaR), E- and D-series resolvins (RvE and RvD), protectins, lipoxins, and precursors thereof such as 18-hydroxy-eicosapentaenoic acid (18-HEPE), 17-hydroxy-docosahexaenoic acid (17-HDHA), and 17,18-epoxyeicosatetraenoic acid (17,18-EEQ), collectively referred to as specialized pro-resolving lipid mediators (SPM). SPM are endogenously formed by lipoxygenases, cyclooxygenase-2, and cytochrome P450 monooxygenases (CYP450), and act as potent agonists of active inflammation resolution, signaling via G-protein coupled receptors at nanomolar concentrations. The effectiveness of SPM against a multitude of infectious and inflammatory diseases has been demonstrated in studies with rodents [6]. For example, RvE1, RvD2, protectin D1 (PD1), and LXA4 enhance the clearance of pathogenic Pseudomonas gingivalis [7], E. coli [8], Herpes simples [9], Candida [10], H5N1 Influenza [11].

LXA4, LXB4, RvE1, RvE3, RvD1-5, RvD2, PD1, MaR1, MaR2 are protective in models of periodontitis, cystic fibrosis, neuroinflammation, ischemic stroke, Alzheimer's disease [12], atherosclerosis [13], non-alcoholic fatty liver disease [14], corneal injury [15], retinopathy [16], glaucoma [17], colitis [18], asthma [19, 20], insulin resistance [14], arthritis [21], and pain [22].

Moreover, several precursors of SPM have themselves been shown to exert pro-resolving effects. For example, 18-hydroxy-eicosapentaenoic acid (18-HEPE) counteracts the development of cardiovascular diseases by inhibiting monocyte adhesion to vascular endothelial cells [23] and by inhibiting pressure overload-induced maladaptive cardiac remodeling [24]. Similarly, 17,18-EEQ has cardio-protective, anti-arrhythmic, vasodilatory, and anti-inflammatory properties [5]. Paracrine secretion of ARA-derived 15-HETE by enteric glial cells supports gut barrier function, a process that is impaired in e.g. Crohn's disease [25].

Translation of these promising preclinical findings towards improving human health has however shown to be challenging. Direct delivery of SPM by intravenous or intraperitoneal injection, as has been done in experimental studies, is not feasible for humans, particularly not in the context of preventive approaches. Oral delivery of SPM- or SPM precursor-containing supplements or foods is not reasonable because of their relatively short half-life in biological fluids, which are therefore unlikely to reach their target tissue. In this regard WO 2017/041094 discloses that a concentrated esterified fish oil contains only ˜0.0005% 18-HEPE and 17-HDHA, and even enrichment of these SPM precursors by supercritical fluid extraction yields not more than 0.05% (18-HEPE+17-HDHA)/total omega-3.

Clinical trials with EPA/DHA have yielded inconclusive or null results, especially for patients with inflammatory bowel diseases, asthma, and traits of the metabolic syndrome [2]. This lack of benefit for humans contrasts with the effective treatment of the respective animal disease models by SPM [6]. We reason that the conversion of omega-3 (and omega-6) to SPM is a crucial step which is decisive for delivering successful outcomes from any interventions aiming to prevent, cure, or treat inflammatory diseases with polyunsaturated fatty acids (PUFA). We also conceive that the SPM-producing machinery is dysfunctional under certain conditions. This idea is supported by findings of reduced (local or circulating) SPM levels in diabetic wounds [26], metabolic syndrome [27], asthma [19, 28], ulcerative colitis [29], Crohn's disease [25], and periodontitis [30], as well as reduced expression or activity of SPM-producing enzymes in severe asthma [28], ulcerative colitis [29], cystic fibrosis [31], periodontitis [30], and Alzheimer's disease [12].

The objective of this invention is therefore to provide a technology that promotes SPM formation inside an organism to provide a benefit to humans and animals suffering from the above-mentioned conditions and that are in need of novel strategies to prevent, ameliorate or cure such and similar conditions, where supplementation of omega-3s alone has yielded little or no success.

This goal is achieved by the invention combining a suitable omega-3 source with a newly discovered microbial SPM-producer in a suitable formulation, such that the combination technology delivers enhanced and/or targeted efficacy compared to an omega-3 source alone. The suitable formulation allows for concomitant release of the components in gastrointestinal sections distal to the small intestine, thereby preventing the omega-3 source from being absorbed in the small intestine and making it more available for metabolization by the microbial SPM-producer.

Biosynthesis of SPM has been described in detail for eukaryotic cells, in particular for granulocytes and monocytes. Macrophages can express all enzymes that are required for SPM biosynthesis; other cell types expressing only selected enzymes can do so together with complementing cells. ALOX5 is found in mast cells, ALOX12 in skin and epithelial cells, ALOX15 in dendritic and enteric glial cells [25], and COX-2 and CYP450 isoforms in epithelial cells.

Given that the gut microbiota determines an individual's response to food ingredients and subsequently modifies health outcomes, we identified it as a target for our technological approach of facilitated endogenous SPM production. Microbiota-targeted strategies in general include the application of prebiotics and probiotics with the intention to modify the composition and activity of the microbiota. Probiotics are live microorganisms, which confer a health benefit on the host when administered in adequate amounts (FAO-WHO; Probiotics in food. Health and nutritional properties and guidelines for evaluation; FAO Food and Nutritional Paper 85, 2006). Prebiotics support the growth of beneficial microorganisms. Prebiotic effects of omega-3 fatty acids have been described [32, 33], but vice versa, possible metabolic impact of gut microbes on omega-3s remained to be determined and are disclosed in this invention. Occurrence of oxygen-consuming enzymes in gut-residing microorganisms is limited; cyclooxygenases and lipoxygenases appear to be absent from gastrointestinal bacteria and archaea, with the exception of a 15-lipoxygenase expressed by pathogenic Pseudomonas aeruginosa [34]. CYP450 monooxygenases have been detected in the Genus Bacillus [35]. CYP102A1, also named CYP450BM-3, is a bifunctional enzyme found in the species Bacillus megaterium that catalyzes the NADPH-dependent hydroxylation of polyunsaturated fatty acids via consecutive oxygenase and reductase activities. This P450 system consists of a polypeptide chain with two different domains, one containing the hemoprotein and the other one containing a FAD-reductase. This bacterial cytochrome P450 class is soluble and obtains the electrons necessary for the reaction mechanism from an NADH-dependent FAD-containing reductase via an iron-sulphur protein of the [2Fe-2S] type [36]. Purified CYP450BM-3 derived from an expression vector construct has been shown to generate 18-HEPE from EPA in a cell-free reaction [37]. However, a possible application of such reaction in a probiotic or synbiotic strategy, wherein a wildtype probiotic strain is the “catalyst” for activation of extracellular EPA/DHA to 18-HEPE or other SPM, and more important, makes these molecules available to the host, has not been described. Furthermore, oxygenation of omega-3 or omega-6 compounds to any other SPM or bioactive lipid mediator by any other probiotic microorganism has thus far not been described.

The present invention is directed to preparation comprising at least one probiotic strain belonging to the genus Bacillus megaterium, and a polyunsaturated fatty acid component comprising at least one omega-3 or omega-6 fatty acid selected from eicosapentaenoic acid (EPA), docosahexaenoic acid (DHA), arachidonic acid (ARA), alpha linolenic acid, stearidonic acid, eicosatetraenoic acid, docosapentaenoic acid, linoleic acid, γ-linolenic acid, wherein the polyunsaturated fatty acid component comprises an omega-3 or omega-6 fatty acid salt.

This new preparation promotes the formation of various SPM in the intestinal lumen, whereby they become available to the host and exert physiological functions therein. The oxygen required for biosynthesis of SPM from EPA/DHA is available in the intestinal lumen: gas in the human rectum reportably contains 0.3-1.8% oxygen [38]. Furthermore, a radial partitioning of intraluminal oxygen, increasing from 1 to 40 mmHg pO2 towards the (vascularized) cecal mucosa, has been described for mice [39], indicating that aerotolerant microbes associated with the mucosa face a relatively oxygen-rich environment that allows for oxygen-dependent biochemical reactions.

Bacteria of the species Bacillus megaterium were found to be especially suitable for this effect. Therefore, the probiotic strain comprises a strain of this species. It is a crucial feature of the invention that the strains lead to extracellular amounts of SPM, which is a prerequisite for eliciting physiological effects on the host. We disclose Bacillus megaterium-dependent production of extracellular SPM at nanomolar levels—whereby some SPM are physiologically active at picomolar levels [6]-, exceeding those reported for human plasma [27, 40, 41] and partly for human milk [42]. According to the present invention, it is both feasible to use whole cells and lysed bacterial cells, encompassing all components of the bacterial cell. Cell extracts may also be used.

Bacillus megaterium has recently been detected in human fecal [43] and saliva [44] samples, showing that these bacteria are residents of the human gut. The invention therefore also covers the use of omega-3 or omega-6 components to promote the formation of various SPM in the gastrointestinal tract by gastrointestinal microbiota through e.g. strains of the species Bacillus megaterium as naturally occurring gut inhabitants.

The cells of the strains of the current invention may be present, in particular in the compositions of the current invention, as spores (which are dormant), as vegetative cells (which are growing), as transition state cells (which are transitioning from growth phase to sporulation phase) or as a combination of at least two, in particular all of these types of cells. Therefore, in a preferred embodiment, the probiotic strain is present in a dormant form or as vegetative cells.

In a preferred embodiment, the omega-3 or omega-6 fatty acids are either in the form of free fatty acids, salts, natural triglycerides, fish oil, phospholipid esters or ethyl esters.

In a further preferred configuration, the fatty acids are selected from the omega-3 fatty acids EPA and DHA or wherein the omega-6 fatty acid component is ARA.

An additional configuration of the present invention is a combination of any of the above-mentioned compositions with 5-Aminolevulinic Acid, a compound that enhances heme biosynthesis [45] and thereby may trigger oxygenase activities of Bacillus megaterium.

In a preferred embodiment the probiotic strain is selected from one or more of the following: Bacillus megaterium DSM 32963, DSM 33296 or DSM 33299.

Bacillus megaterium DSM 32963, DSM 33296 and DSM 33299 have been identified by screening of naturally occurring isolates. They have been deposited with the DSMZ on Nov. 27, 2018 (DSM 32963) and on Oct. 17, 2019 under the provisions of the Budapest Treaty on the International Recognition of the Deposit of Microorganisms for the Purpose of Patent Procedure under the Accession Number as mentioned before in the name of Evonik Degussa GmbH.

Thus, the Bacillus megaterium strain used for the preparation according to the present invention is selected from the following group:

a) One of the Bacillus megaterium strains as deposited under DSM 32963, DSM 33296 and DSM 33299 at the DSMZ;

b) a mutant of the Bacillus megaterium strain as deposited under DSM 32963 having all identifying characteristics of the strain DSM 32963, wherein said mutant preferably has a DNA sequence identity to the strain DSM 32963 of at least 95%, preferably at least 96, 97 or 98%, more preferably at least 99 or 99.5%;

c) a preparation of (a) or (b);

d) a preparation containing an effective mixture of metabolites as contained in (a), (b) or (c).

The Bacillus megaterium strain as deposited under DSM 32963 at the DSMZ exhibits the following characterizing sequences:

a) a 16S rDNA sequence with a sequence identity of at least 99.5%, above all 100%, to the polynucleotide sequence according to SEQ ID NO: 1 or SEQ ID NO: 2;

b) a yqfD sequence with a sequence identity of at least 99.5%, above all 100%, to the polynucleotide sequence according to SEQ ID NO: 3;

c) a gyrB sequence with a sequence identity of at least 99.5%, above all 100%, to the polynucleotide sequence according to SEQ ID NO: 4;

d) an rpoB sequence with a sequence identity of at least 99.5%, above all 100%, to the polynucleotide sequence according to SEQ ID NO: 5;

e) a groEL sequence with a sequence identity of at least 99.5%, above all 100%, to the polynucleotide sequence according to SEQ ID NO: 6.

The Bacillus megaterium strain as deposited under DSM 33296 at the DSMZ exhibits the following characterizing sequences:

a) a 16S rDNA sequence with a sequence identity of at least 99.5%, above all 100%, to the polynucleotide sequence according to SEQ ID NO: 13 or SEQ ID NO: 14;

b) a yqfD sequence with a sequence identity of at least 99.5%, above all 100%, to the polynucleotide sequence according to SEQ ID NO: 15;

c) a gyrB sequence with a sequence identity of at least 99.5%, above all 100%, to the polynucleotide sequence according to SEQ ID NO: 16;

d) an rpoB sequence with a sequence identity of at least 99.5%, above all 100%, to the polynucleotide sequence according to SEQ ID NO: 17;

e) a groEL sequence with a sequence identity of at least 99.5%, above all 100%, to the polynucleotide sequence according to SEQ ID NO: 18.

The Bacillus megaterium strain as deposited under DSM 33299 at the DSMZ exhibits the following characterizing sequences:

a) a 16S rDNA sequence with a sequence identity of at least 99.5%, above all 100%, to the polynucleotide sequence according to SEQ ID NO: 25 or SEQ ID NO: 26;

b) a yqfD sequence with a sequence identity of at least 99.5%, above all 100%, to the polynucleotide sequence according to SEQ ID NO: 27;

c) a gyrB sequence with a sequence identity of at least 99.5%, above all 100%, to the polynucleotide sequence according to SEQ ID NO: 28;

d) an rpoB sequence with a sequence identity of at least 99.5%, above all 100%, to the polynucleotide sequence according to SEQ ID NO: 29;

e) a groEL sequence with a sequence identity of at least 99.5%, above all 100%, to the polynucleotide sequence according to SEQ ID NO: 30.

Thus, a further subject of the current invention is a Bacillus megaterium strain, in particular a B. megaterium strain as mentioned before, exhibiting at least one, preferably all, of the following characteristics:

a) a 16S rDNA sequence with a sequence identity of at least 99%, preferably at least 99.5%, more preferably at least 99.8 or 99.9%, above all 100%, to the polynucleotide sequence according to SEQ ID NO: 1 or SEQ ID NO: 2, SEQ ID NO: 13 or SEQ ID NO: 14 or SEQ ID NO: 25 or SEQ ID NO: 26;

b) a yqfD sequence with a sequence identity of at least 99%, preferably at least 99.5%, more preferably at least 99.8 or 99.9%, above all 100%, to the polynucleotide sequence according to SEQ ID NO: 3, SEQ ID NO: 15 or SEQ ID NO: 27;

c) a gyrB sequence with a sequence identity of at least 99%, preferably at least 99.5%, more preferably at least 99.8 or 99.9%, above all 100%, to the polynucleotide sequence according to SEQ ID NO: 4, SEQ ID NO: 16 or SEQ ID NO: 28.

Preferably, this B. megaterium strain exhibits at least one, more preferably all, of the following further characteristics:

d) a rpoB sequence with a sequence identity of at least 99%, preferably at least 99.5%, more preferably at least 99.8 or 99.9%, above all 100%, to the polynucleotide sequence according to SEQ ID NO: 5, SEQ ID NO: 17 or SEQ ID NO: 29;

e) a groEL sequence with a sequence identity of at least 99%, preferably at least 99.5%, more preferably at least 99.8 or 99.9%, above all 100%, to the polynucleotide sequence according to SEQ ID NO: 6, SEQ ID NO: 18 or SEQ ID NO: 30.

Thus, a particular subject of the current invention is also a Bacillus megaterium strain, exhibiting the following characteristics:

a) a 16S rDNA sequence according to SEQ ID NO: 1 or SEQ ID NO: 2, SEQ ID NO: 13 or SEQ ID NO: 14 or SEQ ID NO: 25 or SEQ ID NO: 26;

b) a yqfD sequence according to SEQ ID NO: 3, SEQ ID NO: 15 or SEQ ID NO: 27;

c) a gyrB sequence according to SEQ ID NO: 4, SEQ ID NO: 16 or SEQ ID NO: 28.

Preferably, this B. megaterium strain exhibits the following further characteristics:

d) an rpoB sequence according to SEQ ID NO: 5, SEQ ID NO: 17 or SEQ ID NO: 29;

e) a groEL sequence according to SEQ ID NO: 6, SEQ ID NO: 18 or SEQ ID NO: 30.

In an advantageous configuration EPA and DHA are either in the form of free fatty acids, salts, natural triglycerides, fish oil, phospholipid esters or omega-3 ethyl esters.

EPA and DHA were effectively transformed by probiotic strains to SPM when they were added as fatty acid salts, whose production and application were disclosed previously. WO2016102323A1 describes compositions comprising polyunsaturated omega-3 fatty acid salts that can be stabilized against oxidation. WO2017202935A1 discloses a method for preparing a composition comprising omega-3 fatty acid salts and amines wherein a paste comprising one or more omega-3 fatty acid(s), one or more basic amine(s) and 20% by weight or less water, based on the total weight of the paste, is kneaded until a homogenous paste is obtained.

Therefore, in a preferred configuration of the present invention the omega-3 component comprises an omega-3 fatty acid amino acid salt, wherein the amino acid is chosen from basic amino acids selected from lysine, arginine, ornithine, histidine, citrulline, choline and mixtures of the same.

In a further preferred configuration, the amino acid is chosen from basic amino acids selected from lysine, arginine, ornithine, choline and mixtures of the same.

It is most preferable to use amino acid salts of lysine.

Another preferred configuration of the present invention are formulations of omega-3 dispersions (presumably liposomes) to further improve bioavailablity to probiotic strains. Such dispersion formulations preferably consist of phospholipid mixtures (e.g. deoiled sunflower lecithin) or defined phospholipids, e.g. Dioleylphospatidylcholine (DOPC). Most preferred forms of such dispersion formulations contain free omega-3 fatty acid salts or free omega-3 fatty acids.

Therefore, in this preferred embodiment the polyunsaturated fatty acid component comprises a preparation comprising a dispersion of at least one phospholipid and at least one omega-3 fatty acid.

In a further preferred embodiment, the polyunsaturated fatty acid component comprises a preparation comprising a dispersion of at least one phospholipid and at least one fatty acid salt of a cation with an anion derived from an omega-3 or omega-6 fatty acid. It is particularly preferred to use omega-3 fatty acids.

In an alternative configuration of the present invention the phospholipid is a deoiled phospholipid comprising a phosphatidylcholine content of greater than 40 weight %, preferably 70 weight %, more preferably greater 90 weight % and a phosphatidylethanolamine content of lower than 5 weight %, preferably lower than 1 weight %.

In an alternative embodiment the phospholipid is a non-hydrogenated phospholipid having an oleic and/or linoleic acid content of greater than 70 weight % of total fatty acids.

In a further preferred configuration of the present invention the mass ratio of phospholipid to fatty acid salt is greater than 0.001, preferably greater than 0.05, more preferably greater than 0.01, more preferably greater than 0.09, most preferably greater than 0.39.

In an alternative embodiment the preparation is in the form of a powder or of a liquid that result in colloidal dispersions with mean particle sizes of smaller than 1 μm, preferably smaller than 500 nm, most preferably smaller than 250 nm when mixed with water at a pH value between pH 6.5 and 7.5.

In another embodiment the components are finely dispersed in each other so that both phospholipid and fatty acid salts are present and detectable in amounts of 100 μg and smaller.

A preferred formulation for enteral delivery of a preparation of this invention is a formulation that provides protection against gastric conditions, or a formulation that provides targeted release of the preparation in the small intestine or a formulation that provides targeted release of the preparation in the large intestine. Therefore, in a preferred embodiment, the preparation comprises a coating for delayed release or enteric or colonic release.

One subject of the present invention is the use of a preparation according to the present invention as a feed or food supplement or its use in foodstuffs. Preferred foodstuffs according to the invention are chocolate products, gummies, mueslis, muesli bars, and dairy products.

A further subject of the current invention is also the use of a preparation of the current invention as a synbiotic ingredient in feed or food products.

A further subject of the present invention is a feed- or foodstuff composition containing a preparation according to the present invention and at least one further feed or food ingredient, preferably selected from proteins, carbohydrates, fats, further probiotics, prebiotics, enzymes, vitamins, immune modulators, milk replacers, minerals, amino acids, coccidiostats, acid-based products, medicines, and combinations thereof.

The feed- or foodstuff composition according to the present invention does also include dietary supplements in the form of a pill, capsule, tablet or liquid.

A further subject of the current invention is a pharmaceutical composition containing a preparation according to the present invention and a pharmaceutically acceptable carrier.

A further subject of the present invention is the use of a preparation for the manufacture of pharmaceutical products.

The preparations according to the present invention, when administered to animals or human beings, preferably improve the health status, in particular gut health, cardiovascular health, cardio-metabolic health, lung health, joint health, eye health, mental health, oral health or immune health of an animal or a human being.

A further subject of the current invention is therefore a composition according to the present invention for improving the health status, in particular gut health, cardiovascular health, cardio-metabolic health, lung health, joint health, eye health, mental health, oral health or immune health of an animal or a human being is part of the present invention.

An advantageous configuration according to the present invention is a composition for improving the health status of an animal or a human being by one or more of the following:
increasing the total amount of the following lipid mediators in the host via their production by gastrointestinal microorganisms:

17-hydroxy-DHA (17-HDHA), 14-hydroxy-DHA (14-HDHA), 13-hydroxy-DHA (13-HDHA), 7-hydroxy-DHA (7-HDHA), 4-hydroxy-DHA (4-HDHA), 18-hydroxy-eicosapentaenoic acid (18-HEPE), 15-hydroxy-eicosapentaenoic acid (15-HEPE), 12-hydroxy-eicosapentaenoic acid (12-HEPE), 11-hydroxy-eicosapentaenoic acid (11-HEPE), 8-hydroxy-eicosapentaenoic acid (8-HEPE), 5-hydroxy-eicosapentaenoic acid (5-HEPE), 15-hydroxy-eicosatetraenoic acid (15-HETE), 12-hydroxy-eicosatetraenoic acid (12-HETE), 11-hydroxy-eicosatetraenoic acid (11-HETE), 8-hydroxy-eicosatetraenoic acid (8-HETE), 5-hydroxy-eicosatetraenoic acid (5-HETE), 9-hydroxyoctadecadienoic acid (9-HODE), 13-hydroxyoctadecadienoic acid (13-HODE), 19Z-docosahexaenoic acid (PDX), protectin D1 (PD1), Aspirin-triggered PD1 (AT-PD1), maresin 1 (MaR1), maresin 2 (MaR2), leukotriene B4 (LTB4), t-LTB4, resolvin D1-5 (RvD1-5), Aspirin-triggered RvD1 (AT-RvD1), resolvin E1 (RvE1), resolvin E3 (RvE3), lipoxin A₄ (LXA₄), lipoxin A₅ (LXA₅), lipoxin B₄ (LXB₄), lipoxin B₅ (LXB₅),
increasing the total amount of EPA in the host,
increasing the total amount of DHA in the host,

A further subject of the current invention is also the use of a preparation of the current invention in topical applications on the skin, the eye, and in the oral cavity using suitable matrices or carriers.

In a preferred configuration, the preparation is loaded on and/or in pre-synthesized multiphase biomaterials comprising nanocellulose, wherein the nanocellulose is bacterially synthesized nanocellulose (BNC) selected from
BNC comprising a network of cellulose fibers or nanowhiskers,
BNC comprising two or more different layers of cellulose fibrils, wherein each layer consists of BNC from a different microorganism or from microorganisms cultivated under different conditions,
BNC comprising of at least two different cellulose networks or
a BNC composite material further comprising a polymer.

Nanocellulose is a term referring to nano-structured cellulose. This may be either cellulose nanocrystal (CNC or NCC), cellulose nanofibers (CNF) also called microfibrillated cellulose (MFC), or bacterial nanocellulose (BNC), which refers to nano-structured cellulose produced by bacteria. BNC is a nanofibrilar polymer produced by strains such as Komagataeibacter xylinus, one of the best bacterial species which given the highest efficiency in cellulose production. BNC is a biomaterial having unique properties such as: chemical purity, excellent mechanical strength, high flexibility, high absorbency, possibility of forming any shape and size due to extraordinary formability and softness and many others. Moreover, the material is vegetarian and vegan and comprises a high moisture content.

The bacterial cellulose is a three-dimensional network and is the carrier to immobilize and trap the microorganism and further substances. The immobilized biologicals (including the microorganisms) are used for the biosynthesis of bioactive metabolites (e.g. antimicrobials, metabolic bioactive) in situ/in vivo, triggered release of the microorganisms and bioactives and/or used as immobilized microfactories for fermentation processes.)

A further subject of the present invention is also the use of a Bacillus megaterium strain in a dormant form or as vegetative cells, exhibiting the following characteristics:

a) a 16S rDNA sequence with a sequence identity of at least 99.5%, above all 100%, to the polynucleotide sequence according to SEQ ID NO: 1 or SEQ ID NO:2, SEQ ID NO:13 or SEQ ID NO:14, SEQ ID NO: 25 or SEQ ID NO: 26; and/or

b) a yqfD sequence with a sequence identity of at least 99.5%, above all 100%, to the polynucleotide sequence according to SEQ ID NO: 3, SEQ ID NO: 15 or SEQ ID NO: 27; and/or

c) a gyrB sequence with a sequence identity of at least 99.5%, above all 100%, to the polynucleotide sequence according to SEQ ID NO: 4, SEQ ID NO: 16, SEQ ID NO: 28; and/or

d) an rpoB sequence with a sequence identity of at least 99.5%, above all 100%, to the polynucleotide sequence according to SEQ ID NO: 5, SEQ ID NO: 17, SEQ ID NO: 29; and/or

e) a groEL sequence with a sequence identity of at least 99.5%, above all 100%, to the polynucleotide sequence according to SEQ ID NO: 6, SEQ ID NO: 18, SEQ ID NO: 30;

for the biotechnological production of SPM.

### WORKING EXAMPLES

### Example 1: The Strains Bacillus megaterium DSM 32963, DSM 33296 and DSM 33299 Each Possess Genetic Sequences for a Cytochrome P450 Monooxygenases (CYP450)

The strains Bacillus megaterium DSM 32963, DSM 33296 and DSM 33299 were isolated each from a soil sample from a pristine garden in east Westphalia. They have been deposited with the DSMZ on Nov. 27, 2018 (DSM 32963) and on Oct. 17, 2019 under the provisions of the Budapest Treaty on the International Recognition of the Deposit of Microorganisms for the Purpose of Patent Procedure under the accession number as mentioned before in the name of Evonik Degussa GmbH.

The genome sequence of B. megaterium DSM 32963 contains a gene [SEQ ID No: 7] encoding a protein with an identity of 97,9% at the amino acid level to P450 BM3 (CYP102A1) of B. megaterium ATCC 14581 (AAA87602.1). This enzyme incorporates both, a P450 oxygenase and a NADPH:P-450 reductase[46]. The natural substrates of P450 BM3 were analyzed to be long chain fatty acids (C12 to C20), which are exclusively hydroxylated at the subterminal positions (ω-1 to ω-3)[47].

Lower sequence similarities on amino acid level ranging from 21,1% to 30,5% on partial sequence hits were observed to sequences identified within the genome sequence of B. megaterium DSM 32963 compared to P450 BM3. These further potential cytochrome genes might have similar functions compared to P450 BM3 [Seq ID No: 8-12] (see table 1).

TABLE 1
BLASTp of P450 BM3 against protein sequences of Bacillus megaterium DSM 32963
(SEQ ID in the hit column refers to the corresponding nucleotide sequence)

| | | | | | | | Function |
|---|---|---|---|---|---|---|---|
| | | E- | | HSP | % | % | based on |
| Query sequence | Hit | value | Score | length | Identity | Gaps | BLAST |
| CYP102_Bm_AAA87602 | DSM | 0 | 5.461,00 | 1.049,00 | 97.9 | 0 | Seq ID 7 |
| | 32963_00731 | | | | | | |
| | [SEQ ID No | | | | | | |
| | 7] | | | | | | |
| CYP102_Bm_AAA87602 | DSM | 8.35E−62 | 558 | 568 | 30.49 | 7.87 | sulfite |
| | 32963_00276 | | | | | | reductase |
| | [SEQ ID No | | | | | | (NADPH) |
| | 8] | | | | | | flavoprotein |
| | | | | | | | alpha- |
| | | | | | | | component |
| CYP102_Bm_AAA87602 | DSM | 4.03E−12 | 161 | 187 | 26.9 | 6.6 | cytochrome |
| | 32963_02107 | | | | | | P450 |
| | [SEQ ID No | | | | | | |
| | 9] | | | | | | |
| CYP102_Bm_AAA87602 | DSM | 1.08E−10 | 149 | 285 | 24.56 | 18.42 | cytochrome |
| | 32963_01384 | | | | | | P450 |
| | [SEQ ID No | | | | | | |
| | 10] | | | | | | |
| CYP102_Bm_AAA87602 | DSM | 2.17E−08 | 130 | 210 | 24.38 | 14.05 | cytochrome |
| | 32963_02065 | | | | | | P450 |
| | [SEQ ID No | | | | | | |
| | 11] | | | | | | |
| CYP102_Bm_AAA87602 | DSM | 5.04E−04 | 94 | 292 | 21.08 | 21.08 | cytochrome |
| | 32963_02258 | | | | | | P450 |
| | [SEQ ID No | | | | | | (BM1) |
| | 12] | | | | | | |

The genome sequence of B. megaterium DSM 33296 contains a gene [SEQ ID No: 19] encoding a protein with an identity of 98,9% at the amino acid level to P450 BM3 (CYP102A1) of B. megaterium ATCC 14581 (AAA87602.1). This enzyme incorporates both, a P450 oxygenase and a NADPH:P-450 reductase[46]. The natural substrates of P450 BM3 were analyzed to be long chain fatty acids (C12 to C20), which are exclusively hydroxylated at the subterminal positions (ω-1 to ω-3)[47].

Lower sequence similarities on amino acid level ranging from 21,5% to 30,7% on partial sequence hits were observed to sequences identified within the genome sequence of B. megaterium DSM 33296 compared to P450 BM3. These further potential cytochrome genes might have similar functions compared to P450 BM3 [Seq ID No: 20-24] (see table 2).

TABLE 2
BLASTp of P450 BM3 against protein sequences of Bacillus megaterium DSM 33296
(SEQ ID in the hit column refers to the corresponding nucleotide sequence)

| | | E- | | HSP | | | |
|---|---|---|---|---|---|---|---|
| Query | Hit | value | Score | length | % Identity | % Gaps | Function |
| CYP102_Bm_AAA87 | DSM | 0.00 | 5.506,00 | 1.049,00 | 98.86 | 0.00 | cytochrome |
| 602 | 33296_54_CDS_D | | | | | | P450 |
| | SM 33296_02126 | | | | | | |
| | [SEQ ID no 19] | | | | | | |
| CYP102_Bm_AAA87 | DSM | 1.48E−61 | 556.00 | 556.00 | 30.70 | 7.72 | sulfite |
| 602 | 33296_54_CDS_D | | | | | | reductase |
| | SM 33296_02261 | | | | | | (NADPH) |
| | [SEQ ID no 20] | | | | | | flavoprotein |
| | | | | | | | alpha- |
| | | | | | | | component |
| CYP102_Bm_AAA87 | DSM | 2.74E−12 | 162.00 | 195.00 | 26.34 | 6.34 | cytochrome |
| 602 | 33296_46_CDS_D | | | | | | |
| | SM_33296_01053 | | | | | | |
| | [SEQ ID no 21] | | | | | | |
| CYP102_Bm_AAA87 | DSM | 1.06E−10 | 149.00 | 272.00 | 24.22 | 17.39 | cytochrome |
| 602 | 33296_70_CDS_D | | | | | | P450 |
| | SM_33296_03886 | | | | | | |
| | [SEQ ID no 22] | | | | | | |
| CYP102_Bm_AAA87 | DSM | 1.26E−8 | 132.00 | 226.00 | 24.91 | 15.85 | cytochrome |
| 602 | 33296_46_CDS_D | | | | | | P450 |
| | SM_33296_01002 | | | | | | |
| | [SEQ ID no 23] | | | | | | |
| CYP102_Bm_AAA87 | DSM | 1.03E−3 | 91.00 | 288.00 | 21.45 | 21.16 | cytochrome |
| 602 | 33296_46_CDS_D | | | | | | P450 |
| | SM_33296_01221 | | | | | | |
| | [SEQ ID no 24] | | | | | | |

The genome sequence of B. megaterium DSM 33299 contains a gene [SEQ ID No: 31] encoding a protein with an identity of 96,1% at the amino acid level to P450 BM3 (CYP102A1) of B. megaterium ATCC 14581 (AAA87602.1). This enzyme incorporates both, a P450 oxygenase and a NADPH:P-450 reductase[46]. The natural substrates of P450 BM3 were analyzed to be long chain fatty acids (C12 to C20), which are exclusively hydroxylated at the subterminal positions (ω-1 to ω-3)[47].

Lower sequence similarities on amino acid level ranging from 20,9% to 30,5% on partial sequence hits were observed to sequences identified within the genome sequence of B. megaterium DSM 33299 compared to P450 BM3. These further potential cytochrome genes might have similar functions compared to P450 BM3 [Seq ID No: 32-37] (see table 3).

TABLE 3
BLASTp of P450 BM3 against protein sequences of Bacillus megaterium DSM 33299
(SEQ ID in the hit column refers to the corresponding nucleotide sequence)

| | | | | | | | Function |
|---|---|---|---|---|---|---|---|
| | | E- | | HSP | | | based on |
| Query sequence | Hit | value | Score | length | % Identity | % Gaps | BLAST |
| CYP102_Bm_AAA87 | DSM | 0.00 | 5.390,00 | 1.049,00 | 96.09 | 0.00 | cytochrome |
| 602 | 33299_122_CDS_D | | | | | | P450 |
| | SM 33299_05006 | | | | | | |
| | [SEQ ID no 31] | | | | | | |
| CYP102_Bm_AAA87 | DSM | 6.77E−62 | 558.00 | 568.00 | 30.49 | 7.87 | sulfite |
| 602 | 33299_58_CDS_D | | | | | | reductase |
| | SM 33299_02626 | | | | | | (NADPH) |
| | [SEQ ID no 32] | | | | | | flavoprotein |
| | | | | | | | alpha- |
| | | | | | | | component |
| CYP102_Bm_AAA87 | DSM | 2.56E−12 | 162.00 | 195.00 | 25.85 | 6.34 | cytochrome |
| 602 | 33299_123_CDS_D | | | | | | P450 |
| | SM 33299_05123 | | | | | | |
| | [SEQ ID no 33] | | | | | | |
| CYP102_Bm_AAA87 | DSM | 1.55E−11 | 156.00 | 272.00 | 25.16 | 17.39 | cytochrome |
| 602 | 33299_15_CDS_D | | | | | | P450 |
| | SM 33299_00711 | | | | | | |
| | [SEQ ID no 34] | | | | | | |
| CYP102_Bm_AAA87 | DSM | 4.26E−8 | 127.00 | 210.00 | 23.65 | 13.28 | cytochrome |
| 602 | 33299_123_CDS_D | | | | | | P450 |
| | SM 33299_05165 | | | | | | |
| | [SEQ ID no 35] | | | | | | |
| CYP102_Bm_AAA87 | DSM | 2.54E−6 | 113.00 | 192.00 | 25.56 | 14.35 | cytochrome |
| 602 | 33299_6_CDS_DS | | | | | | P450 |
| | M 33299_00407 | | | | | | |
| | [SEQ ID no 36] | | | | | | |
| CYP102_Bm_AAA87 | DSM | 7.19E−5 | 101.00 | 280.00 | 20.94 | 21.53 | cytochrome |
| 603 | 33299_87_CDS_D | | | | | | P450 |
| | SM 33299_03167 | | | | | | |
| | [SEQ ID no 37] | | | | | | |

### Example 2: Preparation of Omega-3 Fatty Acid Dispersions with Dioleylphosphatidylcholine (DOPC) in Phosphate Buffer

To prepare formulations of omega-3 fatty acid dispersions 0.8 g of dioleylphosphatidylcholine (DOPC, Lipoid GmbH) were dissolved in 1 ml ethanol. 0.2 g of fish oil (Omega-3 1400, Doppelherz®), omega-3 ethyl ester (PronovaPure® 500:200 EE, BASF) or lysine salt of free omega-3 fatty acid in form of omega-3 lysine salt (AvailOm®, Evonik) were added and dissolved. In the case of free omega-3 fatty acid salt, 20 μl of distilled water were added to dissolve the product completely.

The lysine salt of free omega-3 fatty acid in form of omega-3 lysine salt (AvailOm®, Evonik) contains around 67% of fatty acids and high amounts of the omega-3 fatty acids EPA and DHA and small amounts of the omega-3 fatty acid docosapentaenoic acid and the omega-6 fatty acids arachidonic acid, docosatetraenoic acid and docosaenoic acid isomer.

1 ml of the respective solutions was added dropwise to 20 ml of a 0.1 M phosphate buffer, pH=8, at a temperature of 45° C. and under intense stirring. Afterwards the dispersion was put on ice and sonified for 15 minutes to generate nanometer scale dispersions, presumably liposomes (Branson Sonifier, 100% amplitude, 50% impuls). Finally, the dispersions were sterile filtered through 0.2 μm syringe filters. The resulting dispersions were characterized with regards to particle size in via dynamic light scattering (DLS) measurements (Zetasizer Nano ZS, Malvern). The dispersions contained 40 g/I phospholipids and 10 g/I omega-3 fatty acids or esters.

### Example 3: The Strain B. megaterium DSM 32963 is Able to Produce Intracellularly 18-Hydroxy-Eicosapentaenoic Acid (18-HEPE)

For B. megaterium DSM 32963 an associated intracellularly activity of SPM-producing enzyme(s) could be demonstrated. From 10 ml Luria Bertami broth (LB, Thermo Fisher Scientific) with 0.1% Glucose (LBG) a culture of B. megaterium DSM 32963 was grown for 24 h at 30° C. and 200 rpm in a 100 ml flask. The complete culture was transferred to a 200 ml main culture in LBG. The main culture was grown for 6 h at 30° C. and 200 rpm in a 2 l flask. The cell culture was then harvested in 10 ml portions, the supernatant removed by centrifugation (15 min, 4000 rpm, room temperature) and the cell pellet resuspended in 10 ml LBG and 2 ml of supplements (table 2), respectively. These cultures were incubated in 100 ml shaking flasks for 16 h at 30° C. and 200 rpm.

Different forms of eicosapentaenoic acid (EPA) and docosahexaenoic acid (DHA) sources were added to the B. megaterium DSM 32963 cell cultures to a final concentration of 0.4 g/I in form of omega-3 lysine salt (AvailOm® by Evonik), fish oil (Omega-3 1400 by Doppelherz®), and omega-3 ethyl ester (PronovaPure® 500:200 EE by BASF). These substances were added as sonificated emulsions and as dispersion formulations (preparation described in example 2), respectively.

DOPC formulation without omega-3 fatty acid content, and PBS buffer were used as controls without EPA.

TABLE 2
Supplements, preparation of stock solutions, and
its calculated EPA content (g/l)

| | | EPA content |
|---|---|---|
| | Preparation of | calculated |
| Supplement | stock solution | (g/l) |
| DOPC formulation | undiluted | 0 |
| Omega-3 lysine salt | 0.6 g in 100 ml PBS | 2.04 |
| | buffer | |
| Fish oil | 1 g in 100 ml PBS | 2.04 |
| | buffer | |
| Omega-3 ethyl ester | 0.408 g in 100 ml | 2.04 |
| | PBS buffer | |
| Preparation of omega-3 lysine | 6 ml + 4 ml PBS buffer | 2.04 |
| sal tdispersion with DOPC | | |
| Preparation of fish oil dispersion | undiluted | 2.04 |
| withDOPC | | |
| Preparation of omega-3 ethyl | 4.08 ml + 5.92 ml PBS | 2.04 |
| ester dispersion with DOPC | buffer | |
| PBS buffer | undiluted | 0 |

The supernatants were separated by centrifugation (15 min, 4000 rpm, room temperature), and the cell cultures were then each harvested. Afterwards, the supernatants were diluted with a solvent consisting of a water/acetonitrile mixture (ratio supernatants: solvent was 1:2, solvent composition: 65% H₂O, pH8 and 35% MeCN). Pellets were freeze dried overnight and resuspended in a solvent consisting of a water/acetonitrile mixture (ratio pellet: solvent was 1:2, solvent composition: 65% H₂O, pH8 and 35% MeCN). The cell disruption was carried out in Lysing Matrix tubes (0.1 mm silica spheres) in a Ribolyser.

The cell homogenate (and the diluted supernatant) was filtered and then used for the detection of 18-hydroxy-eicosapentaenoic acid (18-HEPE) by LC/ESI-MS analysis (Agilent QQQ 6420, Gemini 3μ C6-Phenyl) in positive SIM-Mode at m/z 318 as well as the precursor compound EPA at m/z 302.

The addition of EPA, in form of omega-3 lysine salt, omega-3 ethyl ester, omega-3 lysine salt dispersions with DOPC, or fish oil dispersions with DOPC to the B. megaterium cells resulted in a cell associated (=intracellular+extracellular adsorbed) accumulation of 18-HEPE (table 3). By far the highest value of intracellular 18-HEPE was achieved by the addition of omega-3 lysine salt; it was tenfold higher than in the other approaches.

TABLE 3
Concentration of cell associated 18-HEPE (mg/ml) in cellular
extract of Bacillus megaterium DSM 32963 cells

| | Cell associated 18- |
|---|---|
| | HEPE in cellular |
| Supplement added | extract (mg/ml) |
| PBS buffer | 0 |
| DOPC formulation | 0 |
| Omega-3 lysine salt | >1.5 |
| Fish oil | 0 |
| Omega-3 ethyl ester | >0.01 |
| Preparation of omega-3 lysine salt dispersion | >0.15 |
| with DOPC | |
| Preparation of fish oil dispersion with DOPC | >0.10 |
| Preparation of omega-3 ethyl ester dispersion | 0 |
| with DOPC | |

### Example 4: The Synbiotic Combination of Bacillus megaterium DSM 32963 and Dispersion Formulation of Omega-3 Fatty Acid Salt AvailOm® Leads to Extracellular Amounts of 18-HEPE

To investigate the amount of extracellularly appearing 18-HEPE, the B. megaterium DSM 32963 cells were cultivated as described in example 1. The cells were resuspended in 10 ml LBG or LBG containing 9.76 g/I FeSSIF-V2 (biorelevant.com), which is a mixture of taurocholate, phospholipids and other components designed to simulate bile surfactants, and 2 ml of supplements (table 2) were added, respectively. Additionally, the supplements were also added respectively to the different media in shaking flasks without cells and treated under the same conditions (controls). The 18-HEPE concentrations of the culture supernatants and controls were determined after incubation at 16 h, 30° C. and 200 rpm (table 4). It could be shown that the Bacillus megaterium DSM 32963 cells are able to synthesize 18-HEPE from omega-3 lysine salt (AvailOm®) dispersions, which is extracellularly detectable. Of note, the omega-3 4 18-HEPE conversion rate detected by this method is up to 0.075, which exceeds the basal content of 18-HEPE of 0.0005% in an esterified fish oil, disclosed by WO 2017/041094. More importantly, we discovered that the omega-3 lysine salt is converted by Bacillus megaterium strains to a multitude of (final) SPM products at even higher concentrations than 18-HEPE (see example 6), which is of physiological relevance.

TABLE 4
measured 18-HEPE concentrations (mg/l) of
culture supernatants and controls

| | | 18-HEPE content (mg/l) | | |
|---|---|---|---|---|
| | | super- | control | -netto- |
| | bile | natant | without | cellular |
| Supplement added | acids | of culture | cells | produced |
| PBS buffer | − | 0.0 | 0.0 | 0.0 |
| DOPC formulation | − | 0.0 | 0.0 | 0.0 |
| Omega-3 lysine salt | − | >0.2 | >0.3 | 0.0 |
| Fish oil | − | 0.0 | 0.0 | 0.0 |
| Omega-3 ethyl ester | − | 0.0 | 0.0 | 0.0 |
| Preparation of omega-3 lysine | − | >0.5 | >0.1; <0.2 | >0.3 |
| salt dispersion with DOPC | | | | |
| Preparation of fish oil dispersion | − | >0.05 | 0.0 | >0.05 |
| with DOPC | | | | |
| Preparation of omega-3 ethyl | − | 0.0 | >0.1 | 0.0 |
| ester dispersion with DOPC | | | | |
| PBS buffer | + | 0.0 | 0.0 | 0.0 |
| DOPC formulation | + | 0.0 | 0.0 | 0.0 |
| Omega-3 lysine salt | + | >0.4 | >0.4 | 0.0 |
| Fish oil | + | 0.0 | 0.0 | 0.0 |
| Omega-3 ethyl ester | + | 0.0 | 0.0 | 0.0 |
| Preparation of omega-3 lysine | + | >1.2 | >0.5; <1 | >0.2 |
| salt dispersion with DOPC | | | | |
| Preparation of fish oil dispersion | + | 0.0 | 0.0 | 0.0 |
| with DOPC | | | | |
| Preparation of omega-3 ethyl | + | 0.0 | 0.0 | 0.0 |
| ester dispersion with DOPC | | | | |

### Example 5: Biotransformation of EPA by Different Bacillus Species

To investigate the ability of different Bacillus species to produce intracellularly 18-HEPE, cells of different species were cultivated as described in example 1. The cells were resuspended in 10 ml LBG containing 9.76 g/I FeSSIF-V2 (biorelevant.com), which is a mixture of taurocholate, phospholipids and other components designed to simulate bile surfactants, and 1.2 ml of the omega-3 lysine salt dispersion with DOPC were added, respectively. The internal 18-HEPE concentrations of the cells after incubation for 16 hat 30° C. and 200 rpm were determined as described in example 3.

Only the Bacillus megaterium cells were able to synthesize 18-HEPE internally from omega-3 lysine salt (AvailOm®) dispersions, whereby B.subtilis, B. amyloliquefaciens, B. pumilus and B. licheniformis were not.

TABLE 5
Intracellularly measured 18-HEPE content (mg/ml) of
B. megaterium DSM 32963 cells

| | | Internal 18-HEPE content |
|---|---|---|
| Strain | Species | (mg/ml in 10 mg pellet) |
| DSM23778 | B. subtilis | 0 |
| (wildtype 168) | | |
| | B. subtilis | 0 |
| | B. amyloliquefaciens | 0 |
| | B. pumilus | 0 |
| | B. licheniformis | 0 |
| DSM 32963 | B. megaterium | >0.15 |

### Example 6: The Production of SPM by Bacillus megaterium DSM 32963 from Omega-3 Fatty Acid Salt AvailOm® Under Different Culture Conditions

Lipidometabolomics:

Bacterial supernatant samples were subjected to lipid extraction using RP-phase solid phase extraction and subsequently analyzed by ultra performance liquid chromatography ESI tandem mass spectrometry (UPLC-MS/MS) according to a published procedure [48]. Under these conditions approximately 40 different LM, including 5-hydroxy-eicosapentaenoic acid 5-HEPE, 8-HEPE, 11-HEPE, 12-HEPE, 15-HEPE, 18-HEPE, 5-hydroxy-eicosatetraenoic acid (5-HETE), 8-HETE, 11-HETE, 12-HETE, 15-HETE, 4-hydroxy-DHA (4-HDHA), 7-HDHA, 13-HDHA, 14-HDHA, 17-HDHA, lipoxin A₄ (LXA4), LXB4, resolvin E1 (RvE1), RvE3, resolvin D1-5 (RvD1-5), AT-RvD1, RvD2, protectin D1 (PD1), AT-PD1, maresin 1(MaR1), MaR2, plus 4 fatty acid substrates can be detected with a lower limit of detection of 1 pg.

To investigate if Bacillus megaterium is capable of producing other PUFA oxygenation products in addition to 18-HEPE, a lipidometabolomics screening of supernatants from Bacillus megaterium strain 32963 cultured as in example 4 with AvailOm® was performed, either dissolved or in a dispersion formulation, with or without bile acids. Cell-free preparations of AvailOm® formulations were treated and analyzed in parallel and served as controls for non-enzymatic, spontaneous formation of oxygenation products. Values given in table 6 display net concentrations of products formed, i.e. after subtraction of control values. As can be seen, numerous oxygenation products including SPM have been formed by the bacteria. Concentrations of several SPM exceed by far the concentrations of SPM found in human plasma samples, which are typically in a range of ˜20-100 pg/ml [27, 40, 41]. For comparison, human breast milk contains ˜6.000 pg/ml RvE1 and ˜10.000 pg/ml RvD1 [42]. Given the fact that SPM exert receptor-mediated effects in vitro and in vivo in rodents in the nM or even pM range [6], findings displayed in table 6 strongly imply the physiological and therapeutic importance of our invention.

The type of PUFA formulation had a great impact on product levels, which were generally higher in the presence of PUFA dispersion formulations and/or the addition of bile acids as solubilizers. In parallel, abundance of mono-hydroxylated SPM precursors 5-HEPE, 11-HEPE, 12-HEPE, 15-HEPE, 18-HEPE, 5-HETE, 8-HETE, and 9-NODE was lower in these samples compared to samples treated with AvailOm in absence of dispersion formulation and bile acids. This can be explained by an increased conversion of these precursors to di- and trihydroxylated fatty acids.

TABLE 6
Extracellular concentrations of PUFA oxygenation products of
Bacillus megaterium DSM 32963 cells

| | | − bile acids | | + bile acids |
|---|---|---|---|---|
| | − bile acids | AvailOm | + bile acids | AvailOm |
| | AvailOm | dispersion | AvailOm | dispersion |
| Substance | pg/ml | pg/ml | pg/ml | pg/ml |
| 7-HDHA | | | | 397 |
| 18-HEPE | 24463 | | | |
| 15-HEPE | 7536 | | | |
| 12-HEPE | 4413 | | | |
| 11-HEPE | 4659 | | | |
| 5-HEPE | 2253 | | | |
| 8-HETE | | | | 148 |
| 5-HETE | 13 | | 1834 | |
| 9-HODE | 43 | | | |
| PDX 1 | 109 | 19542 | 16623 | 27541 |
| PD1 1 | 113 | 3841 | 3666 | 6564 |
| AT-PD1 1 | 96 | 247 | | |
| RvD1 | 598 | 1863 | | |
| AT-RvD1 | 519 | 4932 | 22 | 732 |
| RvD2 | 87 | 99 | 40 | 27 |
| RvD3 | 178 | 8409 | 1115 | 906 |
| RvD4 | 3681 | | | |
| RvD5 | 68 | 9016 | 4750 | 7071 |
| RvE1 | 88 | 57826 | 22930 | 34786 |
| RvE3 | | 33092 | 769 | 17772 |
| MaR1 | 114 | 145 | 48 | 189 |
| MaR2 | 120 | 197 | | |
| LTB4 | 171 | | 43 | 71 |
| t-LTB4 | 360 | 667 | | |
| LXA4 | | | | |
| LXA5 | 7767 | 41263 | 26620 | 69960 |
| LXB4 | | 76301 | 26356 | 7986 |
| LXB5 | 2376 | 669042 | 299105 | 492508 |

### Example 7: The Production of SPM from a Dispersion Formulation of Omega-3 Fatty Acid Salt AvailOm® by Other Bacillus megaterium Strains

47 additional Bacillus megaterium strains sourced from various habitats were screened for their SPM production capacity to determine if this is a general phenomenon of the species Bacillus megaterium and to detect strain-specific differences in types and quantities of SPM being produced. Cells were cultured as detailed in example 4, with dispersion formulation of AvailOm® serving as omega-3 fatty acid source. Cell-free preparations of AvailOm® were treated and analyzed in parallel and served as controls for non-enzymatic, spontaneous formation of oxygenation products. It was observed that all tested strains produced measurable (>1pg/ml) amounts of various SPM and precursors thereof, that these amounts were hugely different between the strains (up to 2.000 fold), and that concentrations of RvE3 were particularly high (up to 1.3 μg/ml) in most of the strains.

Values given in table 7 display net concentrations of PUFA oxygenation products formed by two of the top performing strains, Bacillus megaterium DSM 33296 and Bacillus megaterium DSM 33299.

TABLE 7
Extracellular concentrations of PUFA oxygenation products of
Bacillus megaterium DSM 33296 and
Bacillus megaterium DSM 33299 cells.

| | Bacillus megaterium | Bacillus megaterium |
|---|---|---|
| | DSM 33296 | DSM 33299 |
| Substance | pg/ml | pg/ml |
| 7-HDHA | 202129 | 243080 |
| 18-HEPE | 391397 | 217725 |
| 15-HEPE | 256797 | 151168 |
| 12-HEPE | 124701 | 79699 |
| 11-HEPE | 124860 | 63306 |
| 5-HEPE | 395188 | 989691 |
| 8-HETE | 24207 | 15941 |
| 5-HETE | 15434 | 9356 |
| PDX 1 | 78345 | 138918 |
| PD1 1 | 99029 | 75342 |
| AT-PD1 1 | 4558 | 10317 |
| RvD1 | 3626 | 4389 |
| AT-RvD1 | 9612 | 48243 |
| RvD2 | 265 | 428 |
| RvD3 | 381 | 941 |
| RvD4 | 1986 | 2523 |
| RvD5 | 15079 | 34168 |
| RvE1 | 5840 | 28379 |
| RvE3 | 1322555 | 880739 |
| MaR1 | 1161 | 1576 |
| MaR2 | 1522 | 1233 |
| LTB4 | 1769 | 2206 |
| t-LTB4 | 1780 | 4181 |
| LXA4 | 1157 | 1430 |
| LXA5 | 4693 | 128825 |

### Example 8: Capsules Comprising EPA-DHA Amino Acid Salts and Bacillus megaterium Strain(s) as Food Supplement or as Drug

The following components were filled in HPMC capsules (size 00).

TABLE 7
Preparations for filling into HPMC capsules.

| Compound | Capsule I | Capsule II | Capsule III |
|---|---|---|---|
| Omega-3 amino acid* | 250 mg | 50 mg | 800 mg |
| salt | | | |
| Bacillus megaterium | 1 × 10⁷ CFU- | 1 × 10⁷ CFU- | 1 × 10⁷ CFU- |
| strain^{#} | 1 × 10¹¹ CFU | 1 × 10¹¹ CFU | 1 × 10¹¹ CFU |

*Amino acids are selected from L-ornithine, L-lysine and L-arginine.
^{#}Strain selected from Bacillus megaterium DSM 32963, DSM 33296, DSM 33299.

The capsules may further contain amino acids selected from L-ornithine, L-aspartate, L-lysine and L-arginine.

The capsules may further contain further carbohydrate ingredients, selected from arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum and xylooligosaccharides.

The capsules may further contain one or more plant extracts, selected from ginger, cinnamon, grapefruit, parsley, turmeric, curcuma, olive fruit, panax ginseng, horseradish, garlic, broccoli, spirulina, pomegranate, cauliflower, kale, cilantro, green tea, onions, and milk thistle.

The capsules may further contain astaxanthin, charcoal, chitosan, glutathione, monacolin K, plant sterols, plant stanols, sulforaphane, collagen, hyalurone, phosphatidylcholine.

The capsules may comprise further vitamins selected from biotin, vitamin A, vitamin B1 (thiamine), vitamin B2 (riboflavin), vitamin B3 (niacin), vitamin B5 (pantothenic acid), vitamin B9 (folic acid or folate), vitamin C (ascorbic acid), vitamin D (calciferols), vitamin E (tocopherols and tocotrienols) and vitamin K (quinones) or minerals selected from sulfur, iron, chlorine, calcium, chromium, cobalt, copper, magnesium, manganese, molybdenum, iodine, selenium, and zinc.

### REFERENCES

1. Balk E M, Lichtenstein A H: Omega-3 Fatty Acids and Cardiovascular Disease: Summary of the 2016 Agency of Healthcare Research and Quality Evidence Review. Nutrients 2017, 9(8).
2. Calder P C: Marine omega-3 fatty acids and inflammatory processes: Effects, mechanisms and clinical relevance. Biochim Biophys Acta 2015, 1851(4):469-484.
3. Papanikolaou Y, Brooks J, Reider C, Fulgoni V L, 3rd: U.S. adults are not meeting recommended levels for fish and omega-3 fatty acid intake: results of an analysis using observational data from NHANES 2003-2008. Nutr J 2014, 13:31.
4. Clarke T C, Black L I, Stussman B J, Barnes P M, Nahin R L: Trends in the use of complementary health approaches among adults: United States, 2002-2012. Natl Health Stat Report 2015(79):1-16.
5. Schunck W H, Konkel A, Fischer R, Weylandt K H: Therapeutic potential of omega-3 fatty acid-derived epoxyeicosanoids in cardiovascular and inflammatory diseases. Pharmacol Ther 2018, 183:177-204.
6. Serhan Conn., Krishnamoorthy S, Recchiuti A, Chiang N: Novel anti-inflammatory—pro-resolving mediators and their receptors. Curr Top Med Chem 2011, 11(6):629-647.
7. Serhan Conn., Jain A, Marleau S, Clish C, Kantarci A, Behbehani B, Colgan S P, Stahl G L, Merched A, Petasis N A et al: Reduced inflammation and tissue damage in transgenic rabbits overexpressing 15-lipoxygenase and endogenous anti-inflammatory lipid mediators. J Immunol 2003, 171(12):6856-6865.
8. Prescott D, McKay D M: Aspirin-triggered lipoxin enhances macrophage phagocytosis of bacteria while inhibiting inflammatory cytokine production. Am J Physiol Gastrointest Liver Physiol 2011, 301(3):G487-497.
9. Rajasagi N K, Reddy P B, Mulik S, Gjorstrup P, Rouse B T: Neuroprotectin D1 reduces the severity of herpes simplex virus-induced corneal immunopathology. Invest Ophthalmol Vis Sci 2013, 54(9):6269-6279.
10. Baillie J K, Digard P: Influenza—time to target the host? N Engl J Med 2013, 369(2):191-193.
11. Morita M, Kuba K, Ichikawa A, Nakayama M, Katahira J, Iwamoto R, Watanebe T, Sakabe S, Daidoji T, Nakamura S et al: The lipid mediator protectin D1 inhibits influenza virus replication and improves severe influenza. Cell 2013, 153(1):112-125.
12. Zhu M, Wang X, Sun L, Schultzberg M, Hjorth E: Can inflammation be resolved in Alzheimer's disease? Ther Adv Neurol Disord 2018, 11:1756286418791107.
13. Kasikara C, Doran A C, Cai B, Tabas I: The role of non-resolving inflammation in atherosclerosis. J Clin Invest 2018, 128(7):2713-2723.
14. Gonzalez-Periz A, Horrillo R, Ferre N, Gronert K, Dong B, Moran-Salvador E, Titos E, Martinez-Clemente M, Lopez-Parra M, Arroyo V et al: Obesity-induced insulin resistance and hepatic steatosis are alleviated by omega-3 fatty acids: a role for resolvins and protectins. FASEB J 2009, 23(6):1946-1957.
15. Jin Y, Arita M, Zhang Q, Saban D R, Chauhan S K, Chiang N, Serhan Conn., Dana R: Anti-angiogenesis effect of the novel anti-inflammatory and pro-resolving lipid mediators. Invest Ophthalmol Vis Sci 2009, 50(10):4743-4752.
16. Connor K M, SanGiovanni JP, Lofqvist C, Aderman C M, Chen J, Higuchi A, Hong S, Pravda E A, Majchrzak S, Carper D et al: Increased dietary intake of omega-3-polyunsaturated fatty acids reduces pathological retinal angiogenesis. Nat Med 2007, 13(7):868-873.
17. Livne-Bar I, Wei J, Liu H H, Alqawlaq S, Won G J, Tuccitto A, Gronert K, Flanagan J G, Sivak J M: Astrocyte-derived lipoxins A₄ and B4 promote neuroprotection from acute and chronic injury. J Clin Invest 2017, 127(12):4403-4414.
18. Arita M, Yoshida M, Hong S, Tjonahen E, Glickman J N, Petasis N A, Blumberg R S, Serhan Conn.: Resolvin E I, an endogenous lipid mediator derived from omega-3 eicosapentaenoic acid, protects against 2,4,6-trinitrobenzene sulfonic acid-induced colitis. Proc Natl Acad Sci USA 2005, 102(21):7671-7676.
19. Barnig C, Frossard N, Levy B D: Towards targeting resolution pathways of airway inflammation in asthma. Pharmacol Ther 2018, 186:98-113.
20. Aoki H, Hisada T, Ishizuka T, Utsugi M, Kawata T, Shimizu Y, Okajima F, Dobashi K, Mori M: Resolvin E1 dampens airway inflammation and hyperresponsiveness in a murine model of asthma. Biochem Biophys Res Commun 2008, 367(2):509-515.
21. Perretti M, Norling L V: Actions of SPM in regulating host responses in arthritis. Mol Aspects Med 2017, 58:57-64.
22. Dalli J, Zhu M, Vlasenko N A, Deng B, Haeggstrom J Z, Petasis N A, Serhan Conn.: The novel 13S,14S-epoxy-maresin is converted by human macrophages to maresin 1 (MaR1), inhibits leukotriene A4 hydrolase (LTA4H), and shifts macrophage phenotype. FASEB J 2013, 27(7):2573-2583.
23. Liu Y, Fang X, Zhang X, Huang J, He J, Peng L, Ye C, Wang Y, Xue F, Ai D et al: Metabolic profiling of murine plasma reveals eicosapentaenoic acid metabolites protecting against endothelial activation and atherosclerosis. Br J Pharmacol 2018, 175(8):1190-1204.
24. Endo J, Sano M, Isobe Y, Fukuda K, Kang J X, Arai H, Arita M: 18-HEPE, an n-3 fatty acid metabolite released by macrophages, prevents pressure overload-induced maladaptive cardiac remodeling. J Exp Med 2014, 211(8):1673-1687.
25. Pochard C, Coquenlorge S, Jaulin J, Cenac N, Vergnolle N, Meurette G, Freyssinet M, Neunlist M, Rolli-Derkinderen M: Defects in 15-HETE Production and Control of Epithelial Permeability by Human Enteric Glial Cells From Patients With Crohn's Disease. Gastroenterology 2016, 150(1):168-180.
26. Tang Y, Zhang M J, Hellmann J, Kosuri M, Bhatnagar A, Spite M: Proresolution therapy for the treatment of delayed healing of diabetic wounds. Diabetes 2013, 62(2):618-627.
27. Barden A E, Mas E, Croft K D, Phillips M, Mori T A: Specialized proresolving lipid mediators in humans with the metabolic syndrome after n-3 fatty acids and aspirin. Am J Clin Nutr 2015, 102(6):1357-1364.
28. Miyata J, Arita M: Role of omega-3 fatty acids and their metabolites in asthma and allergic diseases. Allergol Int 2015, 64(1):27-34.
29. Mangino M J, Brounts L, Harms B, Heise C: Lipoxin biosynthesis in inflammatory bowel disease. Prostaglandins Other Lipid Mediat 2006, 79(1-2):84-92.
30. Wang C W, Colas R A, Dalli J, Arnardottir H H, Nguyen D, Hasturk H, Chiang N, Van Dyke T E, Serhan Conn.: Maresin 1 Biosynthesis and Proresolving Anti-infective Functions with Human-Localized Aggressive Periodontitis Leukocytes. Infect Immun 2015, 84(3):658-665.
31. Ringholz F C, Buchanan P J, Clarke D T, Millar R G, McDermott M, Linnane B, Harvey B J, McNally P, Urbach V: Reduced 15-lipoxygenase 2 and lipoxin A4/leukotriene B4 ratio in children with cystic fibrosis. Eur Respir J 2014, 44(2):394-404.
32. Robertson R C, Seira Oriach C, Murphy K, Moloney G M, Cryan J F, Dinan T G, Paul Ross R, Stanton C: Omega-3 polyunsaturated fatty acids critically regulate behaviour and gut microbiota development in adolescence and adulthood. Brain Behav Immun 2017, 59:21-37.
33. Mujico J R, Baccan G C, Gheorghe A, Diaz L E, Marcos A: Changes in gut microbiota due to supplemented fatty acids in diet-induced obese mice. Br J Nutr 2013, 110(4):711-720.
34. Vance R E, Hong S, Gronert K, Serhan Conn., Mekalanos J J: The opportunistic pathogen Pseudomonas aeruginosa carries a secretable arachidonate 15-lipoxygenase. Proc Natl Acad Sci USA 2004, 101(7):2135-2139.
35. Furuya T, Shibata D, Kino K: Phylogenetic analysis of Bacillus P450 monooxygenases and evaluation of their activity towards steroids. Steroids 2009, 74(12):906-912.
36. Bernhardt R: Cytochromes P450 as versatile biocatalysts. J Biotechnol 2006, 124(1):128-145.
37. Capdevila J H, Wei S, Helvig C, Falck J R, Belosludtsev Y, Truan G, Graham-Lorence S E, Peterson J A: The highly stereoselective oxidation of polyunsaturated fatty acids by cytochrome P450BM-3. J Biol Chem 1996, 271(37):22663-22671.
38. Levitt Md.: Volume and composition of human intestinal gas determined by means of an intestinal washout technic. N Engl J Med 1971, 284(25):1394-1398.
39. Albenberg L, Esipova T V, Judge C P, Bittinger K, Chen J, Laughlin A, Grunberg S, Baldassano R N, Lewis J D, Li H et al: Correlation between intraluminal oxygen gradient and radial partitioning of intestinal microbiota. Gastroenterology 2014, 147(5):1055-1063 e1058.
40. Polus A, Zapala B, Razny U, Gielicz A, Kiec-Wilk B, Malczewska-Malec M, Sanak M, Childs C E, Calder P C, Dembinska-Kiec A: Omega-3 fatty acid supplementation influences the whole blood transcriptome in women with obesity, associated with pro-resolving lipid mediator production. Biochim Biophys Acta 2016, 1861(11):1746-1755.
41. See VHL, Mas E, Prescott S L, Beilin L J, Burrows S, Barden A E, Huang R C, Mori T A: Effects of prenatal n-3 fatty acid supplementation on offspring resolvins at birth and 12 years of age: a double-blind, randomised controlled clinical trial. Br J Nutr 2017, 118(11):971-980.
42. Weiss Ga., Troxler H, Klinke G, Rogler D, Braegger C, Hersberger M: High levels of anti-inflammatory and pro-resolving lipid mediators lipoxins and resolvins and declining docosahexaenoic acid levels in human milk during the first month of lactation. Lipids Health Dis 2013, 12:89.
43. Piewngam P, Zheng Y, Nguyen T H, Dickey S W, Joo H S, Villaruz A E, Glose Kans., Fisher E L, Hunt R L, Li B et al: Pathogen elimination by probiotic Bacillus via signalling interference. Nature 2018, 562(7728):532-537.
44. Al-Thubiani A S A, Maher Y A, Fathi A, Abourehab M A S, Alarjah M, Khan M S A, Al-Ghamdi S B: Identification and characterization of a novel antimicrobial peptide compound produced by Bacillus megaterium strain isolated from oral microflora. Saudi Pharm J 2018, 26(8):1089-1097.
45. Miura M, Ito K, Hayashi M, Nakajima M, Tanaka T, Ogura S: The Effect of 5-Aminolevulinic Acid on Cytochrome P450-Mediated Prodrug Activation. PLoS One 2015, 10(7):e0131793.
46. Ruettinger R T, Wen L P, Fulco A J: Coding nucleotide, 5′ regulatory, and deduced amino acid sequences of P-450BM-3, a single peptide cytochrome P-450:NADPH-P-450 reductase from Bacillus megaterium. J Biol Chem 1989, 264(19):10987-10995.
47. Kang J Y, Kim S Y, Kim D, Kim D H, Shin S M, Park S H, Kim K H, Jung H C, Pan J G, Joung Y H et al: Characterization of diverse natural variants of CYP102A1 found within a species of Bacillus megaterium. AMB Express 2011, 1(1):1.
48. Werz O, Gerstmeier J, Libreros S, De la Rosa X, Werner M, Norris P C, Chiang N, Serhan Conn.: Human macrophages differentially produce specific resolvin or leukotriene signals that depend on bacterial pathogenicity. Nat Commun 2018, 9(1):59.

1. A preparation, comprising:
a probiotic strain of a genus Bacillus megaterium, and
a polyunsaturated fatty acid component comprising an omega-3 or omega-6 fatty acid selected from the group consisting of eicosapentaenoic acid (EPA), docosahexaenoic acid (DHA), arachidonic acid (ARA), alpha linolenic acid, stearidonic acid, eicosatetraenoic acid, docosapentaenoic acid, linoleic acid, and γ-linolenic acid,
wherein the polyunsaturated fatty acid component comprises an omega-3 or omega-6 fatty acid salt.

2. The preparation according to claim 1, wherein the probiotic strain is present in a dormant form or as vegetative cells.

3. The preparation according to claim 1, wherein the omega-3 or omega-6 fatty acids are in a form of free fatty acids, salts, natural triglycerides, fish oil, phospholipid esters or ethyl esters.

4. The preparation according to claim 1, wherein the omega-3 or omega-6 fatty acid is EPA, DHA or ARA.

5. The preparation according to claim 1, further comprising 5-Aminolevulinic Acid.

6. The preparation according to claim 1, wherein the probiotic strain is at least one selected from the group consisting of Bacillus megaterium DSM 32963, Bacillus megaterium DSM 33296, and Bacillus megaterium DSM 33299.

7. The preparation according to claim 1, wherein the polyunsaturated fatty acid component comprises an omega-3 fatty acid salt.

8. The preparation according to claim 1, wherein the polyunsaturated fatty acid component comprises a preparation comprising a dispersion of at least one phospholipid and at least one omega-3 or omega-6 fatty acid.

9. The preparation according to claim 1, further comprising a coating for delayed release or enteric or colonic release.

10. A feed or food supplement, comprising the preparation according to claim 1.

11. A pharmaceutical product, comprising the preparation according to claim 1.

12. A feed- or foodstuff composition comprising:
the preparation according to claim 1 and
an additional feed or food ingredient.

13. The feed- or foodstuff composition according to claim 12, wherein the feed- or foodstuff composition is suitable for improving a health status, in gut health, cardiovascular health, cardio-metabolic health, lung health, joint health, eye health, mental health, oral health or immune health of an animal or a human being.

14. The feed- or foodstuff composition according to claim 13 for improving the health status of an animal or a human being by
increasing a total amount of one or more lipid mediators in a host via their production by gastrointestinal microorganisms wherein the one or more lipid mediators are selected from the group consisting of 17-HDHA, 14-HDHA, 13-HDHA, 7-HDHA, 4-HDHA, 18-HEPE, 15-HEPE, 12-HEPE, 11-HEPE, 5-HEPE, 15-HETE, 12-HETE, 11-HETE, 8-HETE, 5-HETE, 9-HODE, 13-HODE, PDX, PD1, AT-PD1, MaR1, MaR2, LTB4, t-LTB4, RvD1-5, AT-RvD1, RvE1, RvE3, LXA4, LXA5, LXB4, and LXB5,
increasing a total amount of EPA in the host, and/or
increasing a total amount of DHA in the host.

15. A topical application on the skin, the eye, or the oral cavity employing suitable matrices or carriers, the topical application comprising the preparation according to claim 1.

16. The topical application according to claim 15, wherein the preparation is loaded on and/or in pre-synthesized multiphase biomaterials comprising nanocellulose, wherein the nanocellulose is bacterially synthesized nanocellulose (BNC) selected from the group consisting of
BNC comprising a network of cellulose fibers or nanowhiskers,
BNC comprising two or more different layers of cellulose fibrils, wherein each layer consists of BNC from a different microorganism or from microorganisms cultivated under different conditions,
BNC comprising of at least two different cellulose networks, and
a BNC composite material further comprising a polymer.

17. A method, comprising:
biotechnologically producing a specialized pro-resolving lipid mediator with a Bacillus megaterium strain in a dormant form or as vegetative cells, exhibiting
a) a 16S rDNA sequence with a sequence identity of at least 99.5%, above all 100%, to the polynucleotide sequence according to SEQ ID NO: 1 or SEQ ID NO:2, SEQ ID NO:13 or SEQ ID NO:14, SEQ ID NO: 25 or SEQ ID NO: 26;
b) a yqfD sequence with a sequence identity of at least 99.5%, above all 100%, to the polynucleotide sequence according to SEQ ID NO: 3, SEQ ID NO: 15 or SEQ ID NO: 27;
c) a gyrB sequence with a sequence identity of at least 99.5%, above all 100%, to the polynucleotide sequence according to SEQ ID NO: 4, SEQ ID NO: 16, SEQ ID NO: 28;
d) an rpoB sequence with a sequence identity of at least 99.5%, above all 100%, to the polynucleotide sequence according to SEQ ID NO: 5, SEQ ID NO: 17, SEQ ID NO: 29; and/or
e) a groEL sequence with a sequence identity of at least 99.5%, above all 100%, to the polynucleotide sequence according to SEQ ID NO: 6, SEQ ID NO: 18, SEQ ID NO: 30.
